(19) United States
(12) Patent Application Publication
(10) Pub. No.: US 2018/0311184 A1
(43) Pub. Date: Nov. 1, 2018

(54) TOPICAL ANALGESIC PAIN RELIEF FORMULATIONS, MANUFACTURE AND METHODS OF USE THEREOF

(71) Applicant: George Edward Hoag, Storrs, CT (US)

(72) Inventor: George Edward Hoag, Storrs, CT (US)

(21) Appl. No.: 16/000,387

(22) Filed: Jun. 5, 2018

Related U.S. Application Data
(63) Continuation of application No. 15/764,902, filed on Mar. 30, 2018.

(51) Int. Cl. A61K 31/06; A61K 31/352; A61K 31/045; A61K 31/202; A61P 29/00

(52) U.S. Cl. CPC A61K 31/06; A61K 31/352; A61P 29/00; A61K 31/202; A61K 31/045

(57) ABSTRACT

This disclosure relates to natural topical and analgesic pain relief and anti-inflammation compositions and methods to reduce pain and inflammation. This disclosure also relates to the use of non-steroidal anti-inflammatory compounds (NSAIDs) in hydrophilic compositions comprised of synthetic and natural plant extract compounds that are multifunctional TRPM8 ion channel agonists, TRPA1 and TRPV1 ion channel antagonists, CGRP antagonists, and COX-2 inhibitors. In particular, this disclosure relates to a topical analgesic composition comprising at least one NSAID, at least one synthetic or natural plant extract TRPM8 agonist, at least one synthetic or natural plant extract is a TRPA1 antagonist, and at least one fixed plant seed oil containing Omega-3 fatty acids TRPV1 antagonists, and a carrier.

### RELATED APPLICATIONS

This application claims the benefit of U.S. Provisional Application No. 62/234,963, filed Sep. 30, 2015, U.S. Provisional Application No. 62/347,832, filed Jun. 9, 2016, PCT International Application No. PCT/US2016/054474, filed on Sep. 29, 2016, and U.S. patent application Ser. No. 15/764,902, filed on Mar. 30, 2018; all of which are incorporated herein by reference.

### BACKGROUND OF THE DISCLOSURE

### 1. Field of the Disclosure

This disclosure relates to natural topical and analgesic pain relief and anti-inflammation compositions and methods to reduce pain and inflammation. More particularly, this disclosure relates to the use of non-steroidal anti-inflammatory compounds (NSAIDs) in hydrophilic compositions comprised of synthetic and natural plant extract compounds that are multifunctional TRPM8 ion channel agonists, TRPA1 and TRPV1 ion channel antagonists, CGRP antagonists, and COX-2 inhibitors.

### 2. Description of the Related Art

Pain perception is a complex and actively researched field of scientific inquiry. Cutaneous, joint, muscle, headache and gastrointestinal pain is perceived and then transmitted through nerve endings to the brain. Nociception, the sensing of pain, represents encoding and processing of harmful stimuli in the nervous system. The afferent activity in the nervous system produced by specialized free nerve endings referred to as nociceptors or “pain receptors” only respond to tissue damage caused by intense chemical (e.g., chemical burn on the skin), mechanical (e.g., pinching) or thermal (e.g., heat and cold) stimulation.

In vertebrates, the somatosensory system can discriminate small changes in ambient temperature, which activate nerve endings of primary afferent fibers. These thermosensitive nerves are further distinguished into those that detect either innocuous (non-painful) or noxious (painful) temperatures. The mechanism for the perception of cooling sensation of the skin can be ascertained with as little as a 1° C. change in temperature. When the temperature on the skin decreases and approaches 15° C., however, the perception of cold pain is sensed via nociceptors. Thermosensitive afferent nerve fibers that carry impulses from the body to the brain express ion channels of the transient receptor potential (TRP) family and respond at distinct temperature thresholds. This comprises the molecular basis for thermosensation. Transient Receptor Potential channels are a group of ion channels found in cells of many animal cell types. Many of these channels mediate a variety of sensations like she sensations of pain, hotness, warmth or coldness. The main temperature sensors belong to the TRP cation channel family, but the actual mechanisms underlying the marked temperature sensitivity of opening and closing (gating) of these channels is still largely unknown. While activation of various TRP ion channels causes stimuli such as that the result of chemical, mechanical or thermally induced pain, these same TRP ion channels can be inhibited to decrease or eliminate the sensing of pain.

TRP channels represent a heterogeneous system oriented towards environment perception, and participating in sensing visual, gustatory, olfactive, auditive, mechanical, thermal, and osmotic stimuli. The TRP family of channels, currently contains more than 50 different channels. TRP channel gating is operated by both the direct action on the channel by a plethora of exogenous and endogenous physicochemical stimuli. A large amount of evidence shows that TRPA1 ion channel plays a key role in the detection of pungent or irritant compounds, including compounds contained in different spicy foods, such as allyl isothiocyanate (in mustard oil), horseradish, allicin and diallyldisulfide in garlic, cinnamaldehyde in cinnamon, gingerol (in ginger), eugenol (in cloves), methyl salicylate (in wintergreen), menthol (in peppermint), carvacrol (in oregano), thymol (in thyme and oregano). In addition, environmental irritants and industry pollutants, such as acetaldehyde, formalin, hydrogen peroxide, hypochlorite, isocyanates, ozone, carbon dioxide, ultraviolet light and acrolein (a highly reactive α,β-unsaturated aldehyde present in tear gas, cigarette smoke, smoke from burning vegetation, and vehicle exhaust), have been recognized as TRPA1 activators.

Temperature sensing is controlled by voltage-dependent gating in the cold-sensitive channel TRPA1, the cool-sensitive channel TRPM8 (a transient receptor) and the heat sensitive ion channel TRPV1. When temperatures are greater than about 43° C. and below about 15° C., in addition to temperature sensing there is a feeling of pain. In mammals, six thermosensitive ion channels have been reported, all of which belong to the TRP superfamily. These include TRPV1 (VR1), TRPV2 (VRL-1), TRPV3, TRPV4, TRPM8 (CMR1), and TRPA1 (formerly ANKTM1). These channels exhibit distinct thermal activation thresholds (>43° C. for TRPV1, >52° C. for TRPV2, >˜34-38° C. for TRPV3, >˜27-35° C. for TRPV4, <˜25-28° C. for TRPM8, and <17° C. for TRPA1. Therefore, it is apparent that activating TPRA1 will cause cold pain and TRPV1 and TRPV2 will cause hot pain.

TRPA1 is a TRP channel that functions as a receptor for noxious cold temperatures and various tissue and skin irritations, for example those caused by parabens and burns from alkaline compounds, as well as cooling compounds, such as menthol, as well as methyl salicylate. Several of the TRPA1 activators are also known as triggers of migraine attack. Because TRPA1 is an excitatory ion channel targeted by cold nociception and inflammatory pain, TRPA1 is a promising target for use in identifying analgesic drugs that could inhibit TRPA1.

TRPM8 is a thermosensitive receptor that detects cool temperatures, as well as several essential oil compounds including menthol, 1,8-cineole, geraniol, linalool, thymol, borneol, 2-methylisoborneol, fenchyl alcohol and hydroxycitronellal, as well as the synthetic organic compound icilin. Menthol, in the form of peppermint essential oil, has been used since ancient times for pain relief, which is now known through the TRPM8 activation mechanism. However, menthol has recently been shown to have different effects on TRPA1 gating in humans than in mice. Past research identified that menthol exhibited bimodal action ion channel gating by menthol of mouse TRPA1 (mTRPA1), in which submicromolar to low micromolar-concentrations of menthol caused robust channel activation, but higher concentrations led to a reversible channel block. However, this bimodal action is not observed on human TRPA1 (hTRPA1) because high doses of menthol cause sensory irritation the result of TRPA1 activation. Camphor, another essential oil component, is now known to exert analgesic effects probably through inhibition of TRPA1 and activation of TRPM8. However, camphor is not suited for use as an analgesic compound in this present disclosure because it also causes a warm and hot sensation through TRPV1 and TRPV3 activation. Camphor is also a known antagonist to menthol activated TRPM8. In humans menthol alone has been shown to be a TRPM8 agonist (desirable for a topical analgesic) but also a TRPA1 agonist (undesirable for a topical analgesic because this induces pain).

In recent research, Takaishi, M., Fujita, F., Uchida, K., Yamamoto, S., Sawada Shimizu, M., Hatai Uotsu C. (2012) 1,8-Cineole, a TRPM8 agonist, is a novel natural antagonist of human TRPA1. Mol Pain. 2012; 8:86-97, it was concluded that an effective analgesic compound would activate TRPM8 (i.e., desirable cooling, reducing pain and/or anti-inflammatory properties) and inhibit TRPA1 (i.e., undesirable causation of cold pain and inflammation) but not activate TRPV1 (i.e., undesirable causation of hot pain and inflammation). These researchers reported that 1,8-cineole (eucalyptol) activates human TRPM8 (hTRPM8) without activating hTRPA1. They also demonstrated that 1,8-cineole did not activate hTRPV1 or hTRPV2. 1,8-cineole is present in Eucalyptus oil from several species in highly varying concentrations (less than 5 percent to greater than 80 percent), in several Rosmarinus officinalis chemotypes (up to ˜50 percent) and in Salvia lavandulifolia (up to ˜25 percent). It has been shown that TRPM8 activation decreases inflammation and pain. While TRPM8 activation by menthol was reported by these researchers, it did not decrease human inflammatory response, likely because it also activated TRPA1, which causes inflammation. Further, application of menthol with 1,8-cineole significantly reduced irritation probably through inhibition of TRPA1 by 1,8-cineole.

As a follow-up to this research, an additional study was published by the same research group (Takaishi, M., Uchida, K., Fujita, F., Tominaga, M. (2014) Inhibitory effects of monoterpenes on human TRPA1 and the structural basis of their activity. J Physiol Sci. 64:47-57) on the role of several monoterpene analogs of camphor and their ability to inhibit hTRPA1. They reported that 1,8-cineole, camphor, borneol, 2-methylosoborneol, norcamphor and fenchyl alcohol did not activate hTRPA1 and that borneol, 2-methylosoborneol and fenchyl alcohol at 1 mM completely inhibited hTRPA1 activation by menthol and allyl isothiocyanate (mustard oil) at 1 mM and 10 uM, respectively. It was found that TRPA1 activation by 20 uM AITC was inactivated (IC-50 concentration) in order from lowest to highest concentration by 2-methylosoborneol (0.12 mM), borneol (0.20 mM), fenchyl alcohol 0.32 mM, camphor (1.26 mM) and 1,8-cineole (3.43 mM).

Matta, J. A., Miyares, R. L. & Ahern, G. P. (2007), TRPV1 is a novel target for omega-3 polyunsaturated fatty acids. J. Physiol. 578, 397-411, reported that Omega-3 polyunsaturated fatty acids were novel targets for TRPV1. Specifically they reported that docosahexaenoic acid exhibits the greatest efficacy as a TRPV1 agonist. However, eicosapentaenoic acid and linolenic acid were markedly more effective inhibitors. Comparatively eicosapentaenoic acid but not docosahexaenoic acid profoundly reduced capsaicin-evoked pain-related behavior in mice. Very recently, Morales-Lázaro et al (2016). Inhibition of TRPV1 channels by a naturally occurring omega-9 fatty acid reduces pain and itch. Nature Communications, (7) 13092, reported a naturally occurring monounsaturated fatty acid, oleic acid, inhibited TRPV1 activity, and also pain and itch responses in mice by interacting with the vanilloid (capsaicin)-binding pocket and promoting the stabilization of a closed state conformation.

Natural and synthetic sources of borneol exist. Natural sources of borneol are preferred, including Thymus satureioides (Red Thyme Borneol Type Morrocco) and Cinnamomum burmanni (Mei Pian Tree).

Recently, researchers have reported that in addition to its antinociceptive action and anti-inflammatory role, TRPM8, is a promising therapeutic target for treating internal inflammatory diseases such as colitis and inflammatory bowel disease.

The plant genus Cannabis is a member of the plant family Cannabaceae, and there are 3 primary cannabis species which vary in their biochemical constituents: Cannabis sativa, Cannabis indica, and Cannabis ruderalis. In general, cannabis that has high levels of the psychoactive cannabinoid, delta9-tetrahydrocannabinol (Δ9-THC), and low levels of the non/antipsychoactive cannabinoid, cannabidiol (CBD), is referred to as “marijuana”. Cannabis that has high levels of CBD, and very low insignificant levels of Δ9-THC, is referred to as “industrial hemp,” or “hemp,” and has no psychoactive effects (Baron, et al., 2015). Recent advancements in selective breeding has enabled Cannabis sativa L. to yield high concentrations of CBD and low concentrations of THC. CBD concentrations in these hybrid Cannabis sativa L. plants enable CBD yields of 15% and higher and THC yields of 1% and lower. A breakthrough in the understanding of how cannabis works in the brain occurred with the discovery of the endogenous cannabinoids and receptors. The endocannabinoid system is widely distributed throughout the brain and spinal cord, and plays a role in many regulatory physiological processes including inflammation and nociception/pain. The endocannabinoid system consists of the cannabinoid 1 (CB1) and 2 (CB2) receptors, the endogenous cannabinoid receptor ligands (endogenous cannabinoids) n-arachidonoylethanolamine (anandamide, or AEA) and 2-arachidonoylglycerol (2-AG), as well as their degrading enzymes fatty acid amide hydrolase (FAAH) and monoacylglycerol lipase, respectively. Mechoulam (2013) reported that the CB1 receptor mediated anti-inflammatory effects of cannabinoids are suspected to be secondary to inhibition of arachidonic acid conversion by cyclooxygenase although CB2 receptor activation induces immunosuppression, which also reduces inflammation. Because of this, cannabinoid compounds including Δ9-THC and CBD have become of interest in pain and topical pain relief. This is particularly true of CBD, which exhibits no psychoactive effects.

U.S. Pat. No. 6,949,582 B1 teaches a method of relieving analgesia and reducing inflammation using a cannabinoid delivery topical liniment composition containing from about 97.5% to about 99.5% by weight a 70% monohydric alcohol solution, and from about 0.5% to about 2.5% by weight of a synergistic cannabinoid mixture extracted from the female plant Cannabis sativa L, including in combination: 9-Tetrahydrocannabinol (delta-9-THC), 9-THC Propyl Analogue (THC-V), Cannabidiol (CBD), Cannabidiol Propyl Analogue (CBD-V), Cannabinol (CBN), Cannabichromene (CBC), Cannabichromene Propyl Analogue (CBC-V), Cannabigerol (CBG), terpenoids, and flavonoids. In U.S. Pat. No. 6,949,582 B1, it is taught that the liniment is applied topically, preferably by spraying, and the constituents of the mixture are absorbed through the skin and interact with cannabinoid receptors in the body and tissues of a human patient to produce therapeutic analgesic and anti-inflammatory effects, without undesirable psychotropic side effects. U.S. Publication No. 2015/0086494 A1 teaches a topical formulation comprising a Cannabis derived botanical drug product, wherein the concentration of tetrahydrocannabinol and/or cannabidiol in the topical formulation is greater than 2 milligrams per kilogram. In some embodiments of U.S. Publication No. 2015/0086494 A1, the topical formulation further comprises an analgesic agent, including methyl salicylate, codeine, morphine, methadone, pethidine, buprenorphine, hydromorphine, levorphanol, oxycodone, fentanyl, and a non-steroidal anti-inflammatory drug. The amount of the analgesic agent in the topical formulation is not particularly limited, so long as it is a therapeutically effective amount. A preferred amount is from 0.01 to 5 wt %, relative to the total amount of the topical formulation, more preferably from 0.1 to 1 wt %, relative to the total amount of the topical formulation.

However, research reported by De Petrocellis L. et al., Effects of cannabinoids and cannabinoid-enriched Cannabis extracts on TRP channels and endocannabinoid metabolic enzymes. Br J Pharmacol 2011; 163: 1479-1494, reported that some TRP channels may serve as ionotropic cannabinoid receptors, which, in the context of primary afferent nerve fibers, may contribute to inflammatory hypersensitivity or vasodilatation. These results were supported by their earlier work in which they found previously that some cannabinoids activate TRPV1 (Ligresti et al., 2006) and TRPA1 (De Petrocellis L. et al., Plant-derived cannabinoids modulate the activity of transient receptor potential channels of ankyrin type-1 and melastatin type-8. J Pharmacol Exp Ther 2008; 325:1007-1015) and antagonize TRPM8 (De Petrocellis et al., 2008, supra). Because TRPA1 ion channel is a pain and inflammation sensor, the activation of TRPA1 by cannabinoids, including delta-9-THC and CBD is not a favorable property of cannabinoid compounds. Further because TRPM8 is an ion channel associated with pain relief and anti-inflammation, its blocking by cannabinoid compounds is not a favorable property of cannabinoid compounds, particularly delta-9-THC and CBD.

The present disclosure provides many advantages, which shall become apparent as described below.

### SUMMARY OF THE DISCLOSURE

This disclosure relates in part to compositions and methods of use and manufacture of topical analgesics that are more effective than existing compositions and products. This disclosure also relates to compositions and methods of use and manufacture of analgesics that can be administered both orally and topically.

One feature of this present disclosure is to block the signaling of pain from the skin, muscles and joints to the brain through TRPM8 activation and TRPA1 inactivation without activating TRPV ion channels. Another complimentary feature of this disclosure of is to inhibit the inflammation enzyme cyclo-oxygenase-2 (COX-2). COX-2 is responsible for the formation of a group of inflammatory mediators known as prostaglandins. COX-2 inhibitors have analgesic and anti-inflammatory activity by blocking the transformation of arachidonic acid into prostaglandin H2 selectively. For example, aspirin (acetylsalicylic acid) is a competitive active site inhibitor of COX-2, thereby stopping the formation of prostaglandins and because of this acts as an anti-inflammatory agent. Acetylsalicylic acid is a prodrug in that it is hydrolyzed to salicylic acid which is responsible for the COX inhibition properties of aspirin. Methyl salicylate is the major component in wintergreen essential oil. Methyl salicylate is metabolized in humans to salicylic acid, including following absorption through the skin and is therefore also a prodrug to salicylic acid, a known COX-2 inhibitor. Therefore, methyl salicylate acts like a local-topical application of an aspirin-like compound. It is known that menthol increases the rate of methyl salicylate absorption through the skin, but also decreases the hydrolysis rate of methyl salicylate to salicylic acid.

Yet another feature of this disclosure is the neutralization of the release of calcitonin gene related peptide (CGRP) through reactivation of TRPA1. CGRP is a member of the calcitonin family of peptides, existing in two forms: α-CGRP and β-CGRP. CGRP is produced in both peripheral and central neurons and is a peptide vasodilator and functions in pain transmission. When synthesized in the ventral horn of the spinal cord, CGRP is mainly derived mainly from cell bodies of motor neurons and may contribute to the regeneration of nervous tissue after injury. CGRP is also derived from dorsal root ganglion when synthesized in the dorsal horn of the spinal cord and may be linked to the transmission of pain. CGRP is therefore invoked in the nociception process which contributes to the perception of pain.

This disclosure also relates in part to the incorporation of Essential Fatty Acids (EFAs) in the composition to provide further anti-inflammatory and rapid skin penetration of other bioactive compounds in this present disclosure. Flax seed, pumpkin seed, hemp, hemp seed, chia seed oil, algal oil, perilla seed oil and walnut seed oils high in Omega-3 fatty acids are particularly preferred natural sources of Omega-3 fatty acids featured is this present disclosure.

This disclosure further relates in part to compositions and methods to reduce pain and inflammation additionally comprise a topically active NSAID, for example but not limited to diclofenac or a salt of diclofenac thereof. Compositions of NSAIDs in combination with the TRPA1 antagonists and TRPM8 agonists in this present disclosure are unexpectedly effective in pain relief and reduction of inflammation and methods to treat pain and inflammation. In yet another embodiment of this present disclosure methyl salicylate and a NSAID are combined with the TRPA1 antagonists and TRPM8 agonists in this present disclosure and are unexpectedly effective in pain relief and reduction of inflammation and methods to treat pain and inflammation.

This disclosure yet further relates in part to cannabinoid (both phytocannabinoid and synthetic cannabinoid) compounds, including but not limited to: 9-Tetrahydrocannabinol (delta-9-THC), 9-THC Propyl Analogue (THC-V), Cannabidiol (CBD), Cannabidiol Propyl Analogue (CBD-V), Cannabinol (CBN), Cannabichromene (CBC), Cannabichromene Propyl Analogue (CBC-V), Cannabigerol (CBG), cannabinoid terpenoids, and cannabinoid flavonoids; cannabinol (CBN) are combined with TRPA1 antagonists and optionally, TRPM8 agonists; and further optionally with one or more NSAIDS and optionally oils high in Omega-3 fatty acids. Because of its lack of psychoactive properties CBD is a preferred phytocannabinoid in this disclosure.

This disclosure yet further relates to cannabinoid compounds that are extracts from various portions of Cannabis sativa, Cannabis indica, and Cannabis ruderalis plants. The extracts may be in the form of one or more of powders, resins, solids, oils or granules. The extracts may be single cannabinoid compound extracts or contain multiple cannabinoid compounds.

This disclosure also relates in part to the incorporation of the composition into either a hydrophilic or hydrophobic base, suspensions, solids, semi-solids, powders, or nanoparticles for pharmaceutical compositions, in which the pharmaceutical composition comprises one or more of a sprayable liquid, a gel, a lotion, a film, an ointment, a massage oil, a cream, a paste, a stick, a patch, tablets, capsules, powders or granules. The sprayable liquid may be applied from a hand-pumped spray bottle or alternatively, or an aerosol from an inert gas pressurized container spray.

This disclosure further relates to the manufacture of pharmaceutical compositions comprising solids, semi-solids, powders and granules with one or more excipients selected from the group consisting of diluents, fillers, binders, adhesives, disintegrants, lubricants, anti-adhesives glidents, coloring agents, sweeteners, coating agents, plasticizers, wetting agents, buffers lactose, dibasic calcium phosphate, sucrose, corn (maize) starch, microcrystalline cellulose or modified cellulose (for example hydroxypropyl methylcellulose and hydroxyethylcellulose).

Activation of various TRP ion channels cause stimuli such as that the result of chemical, mechanical or thermally induced pain. It is also known that these same TRP ion channels can be inhibited to decrease or eliminate the sensing of pain. Surprisingly, it has been found in this present disclosure that natural compounds can be combined to control gating to inhibit key pain inducing TRP ion channels, including TRPA1 and TRPV1, and to activate the TRPM8 ion channel. The control of TRP ion channels is a key embodiment in compositions used in this present disclosure to manufacture topical analgesic compositions and serve as the basis of methods to reduce inflammation and pain. Compositions of this present disclosure may include natural fixed seed oils containing high concentrations of Omega-3 essential fatty acids selected from a group comprising: flaxseed oil, hemp oil, hemp seed oil, kiwifruit seed oil, pumpkin seed oil, chia seed oil, algal oil, perilla seed oil and walnut oil. These novel topically administrated compositions and methods relieve inflammation and pain in mammals associated with one or more of nociceptive, neuropathic, somatic pain, radicular pain and associated musculoskeletal, osteoarthritic, muscle, joint, arthritis, rheumatoid arthritis, back, strains and sprains pain associated with sports injuries, post-surgical conditions and other diseases.

Compositions in this present disclosure include menthol, 1,8-cineole and Omega-3 essential fatty acids. Additional compositions include menthol, 1,8-cineole and/or optionally borneol and Omega-3 essential fatty acids. Yet further compositions of this present disclosure 1,8-cineole and/or borneol with or without Omega-3 essential fatty acids. Yet further compositions of this present disclosure include 1,8-cineole and/or borneol with or without Omega-3 essential fatty acids and also with and without methyl salicylate. Yet further compositions of this present disclosure include 1,8-cineole and/or borneol with or without Omega-3 essential fatty acids and also with and without a NSAID. Yet further compositions of this present disclosure include menthol and/or 1,8-cineole and borneol and methyl salicylate. Yet further compositions of this present disclosure include menthol and/or 1,8-cineole and borneol and methyl salicylate and Omega-3 essential fatty acids. These are complementary bioactive natural compounds are used in combination in this present disclosure. Menthol is used as an effective TRPM8 agonist, however in humans it also is a TRPA1 agonist associate with pain and inflammation. 1,8-cineole and borneol are fortuitously naturally occurring bioactive compounds that are TRPA1 antagonists as well as a TRPM8 agonists. 1,8-cineole and borneol therefore inhibit pain and inflammation associated with activation of TRPA1 caused by injuries, pain or by menthol and additionally activates the TRPM8 ion channel. Further it has been surprisingly found that the addition of at least one fixed plant seed oil containing high concentrations of Omega-3 essential fatty acids also serves to reduce pain and inflammation and makes the composition of menthol and 1,8-cineole and/or borneol less irritating to the skin, as well as facilities transport of the topical pain relief compositions through and into the dermis, epidermis, subcutis and to tissues below the skin.

Yet in another embodiment of this present disclosure, cannabinoid (both phytocannabinoid and synthetic cannabinoid) compounds, including but not limited to: 9-tetrahydrocannabinol (delta-9-THC), 9-THC Propyl Analogue (THC-V), Cannabidiol (CBD), Cannabidiol Propyl Analogue (CBD-V), Cannabinol (CBN), Cannabichromene (CBC), Cannabichromene Propyl Analogue (CBC-V), Cannabigerol (CBG), cannabinoid terpenoids, and cannabinoid flavonoids; cannabinol (CBN) are combined with TRPA1 anatagonists and optionally, TRPM8 agonists; and further optionally with one or more NSAIDs and optionally oils high in Omega-3 fatty acids. Because of its lack of psychoactive properties CBD is a preferred phytocannabinoid in this disclosure. Compositions in this present disclosure include menthol, 1,8-cineole, a cannabinoid or mixture of cannabinoid compounds and Omega-3 essential fatty acids. Additional compositions include menthol, 1,8-cineole and/or optionally borneol, Omega-3 essential fatty acids and a cannabinoid or mixture of cannabinoid compounds. Yet further compositions of this present disclosure 1,8-cineole and/or borneol, and a cannabinoid or mixture of cannabinoid compounds with or without Omega-3 essential fatty acids. Yet further compositions of this present disclosure include 1,8-cineole and/or borneol with or without Omega-3 essential fatty acids and also with and without methyl salicylate and a cannabinoid or mixture of cannabinoid compounds. Yet further compositions of this present disclosure include 1,8-cineole and/or borneol with or without Omega-3 essential fatty acids and also with and without a NSAID and a cannabinoid or mixture of cannabinoid compounds. Yet further compositions of this present disclosure include menthol and/or 1,8-cineole and borneol, a cannabinoid or mixture of cannabinoid compounds and methyl salicylate. Yet further compositions of this present disclosure include menthol and/or 1,8-cineole borneol, methyl salicylate, Omega-3 essential fatty acids and a cannabinoid or mixture of cannabinoid compounds.

Accordingly, this present disclosure provides topical analgesic compositions that are administered for the treatment of pain and inflammation associated with nociceptive, neuropathic, somatic pain, radicular pain and methods for reducing such pain in mammals.

This disclosure further relates in part to the incorporation of the composition into either a hydrophilic or hydrophobic base for use as a sprayable liquid, a gel, an ointment, a massage oil, a cream, a stick or a patch. The sprayable liquid may be applied from a hand-pumped spray bottle or alternatively, or an aerosol from an inert gas pressurized container spray. Yet another aspect of this present disclosure is a composition that includes a TRPA1 antagonist, a TRPM8 agonist and a NSAID or optionally a cannabinoid compound, a salicylate, methyl salicylate or acetylsalicylic acid.

These compositions can be applied, for example once, twice, or even four times per day to skin that is unbroken and not bleeding.

In one embodiment of the present disclosure, the compositions and methods to reduce pain and inflammation optionally comprise methyl salicylate which is a bioactive compound that undergoes biotransformation in mammals to salicylic acid following absorption through the skin. Methyl salicylate therefore is a prodrug to salicylic acid, a known COX-2 inhibitor. COX-2 inhibitors prevent inflammation and pain and in one embodiment of this present disclosure are unexpectedly effective as a result of the synergy of methyl salicylate in combination with TRPA1 and TRPV1 antagonists and TRPM8 agonists. The addition of 1,8-cineole and/or borneol in this present disclosure also are effective at inactivating the gating of the TRPA1 ion channel associated with methyl salicylate.

In yet another embodiment of this present disclosure, compositions and methods to reduce pain and inflammation additionally comprise a topically active NSAID, for example but not limited to diclofenac or a salt of diclofenac thereof. Compositions of NSAIDs in combination with the TRPA1 and TRPV1 antagonists and TRPM8 agonists in this present disclosure are unexpectedly effective in pain relief and reduction of inflammation and methods to treat pain and inflammation. In yet another embodiment of this present disclosure methyl salicylate and a NSAID are combined with the TRPA1 and TRPV1 antagonists and TRPM8 agonists in this present disclosure and are unexpectedly effective in pain relief and reduction of inflammation and methods to treat pain and inflammation.

The pharmaceutical compositions of a NSAID and optionally methyl salicylate combined with the TRPA1 and TRPV1 antagonists and TRPM8 agonists in this present disclosure comprise a therapeutically effective amount of a NSAID to reduce nociceptive, neuropathic, somatic pain, radicular pain and inflammation associated with musculoskeletal, osteoarthritic, muscle, joint, arthritis, rheumatoid arthritis, back, strains and sprains pain associated with sports injuries and other diseases or trauma. The compositions can be applied, for example once, twice, or even four times per day to skin that is unbroken and not bleeding.

The pharmaceutical compositions of the NSAID diclofenac (or its sodium salt) and optionally methyl salicylate combined with the TRPA1 antagonists and TRPM8 agonists in this present disclosure and optionally TRPV1 antagonists Omega-3 essential fatty acids comprise a therapeutically effective amount of a NSAID to reduce nociceptive, neuropathic, somatic pain, radicular pain and inflammation associated with musculoskeletal, osteoarthritic, muscle, joint, arthritis, rheumatoid arthritis, back, strains and sprains pain associated with sports injuries and other diseases or trauma. The compositions can be applied, for example once, twice, or even four times per day to skin that is unbroken and not bleeding.

A topically active NSAID is meant as a NSAID when used in combination with a suitable carrier can be transported through the skin barrier of mammals and becomes locally active in and below the skin and is safe for exposure to skin without unacceptable reactions.

The activation of certain ion channels (agonists) that reduce pain and/or inflammation, and the deactivating of other ion channels (antagonists) that cause pain and/or inflammation are key components in this present disclosure.

Because TRPA1 and TRPV1 both cause pain, it is a key feature in this present disclosure to minimize and/or eliminate activation of these ion channels.

The elimination of TRPA1 activation by menthol is also a key feature of this present disclosure.

Natural and synthetic sources of borneol exist and both can be used in this disclosure as hTRPA1 antagonists. Natural sources of borneol are preferred, including Thymus satureioides (Red Thyme Borneol Type Morrocco) and Cinnamomum burmanni (Mei Pian Tree).

Further objects, features and advantages of the present disclosure will be understood by reference to the following drawings and detailed description.

### BRIEF DESCRIPTION OF THE DRAWINGS

FIG. 1 shows the composition of a topical analgesic in the form of a sprayable liquid or aerosol with methyl salicylate in accordance with Example 1.

FIG. 2 shows the composition of a topical analgesic in the form of a sprayable liquid or aerosol without methyl salicylate in accordance with Example 2.

FIG. 3 shows the composition of a topical analgesic in the form of a gel with methyl salicylate in accordance with Example 3.

FIG. 4 shows the composition of a topical analgesic in the form of a gel without methyl salicylate in accordance with Example 4.

FIG. 5 shows the composition of a topical analgesic in the form of a cream with methyl salicylate in accordance with Example 5.

FIG. 6 shows the composition of a topical analgesic in the form of a cream without methyl salicylate in accordance with Example 6.

FIG. 7 shows the composition of a topical analgesic in the form of a wax with methyl salicylate in accordance with Example 7.

FIG. 8 shows the composition of a topical analgesic is the form of a wax without methyl salicylate in accordance with Example 8.

FIG. 9 shows the composition of a topical analgesic in the form of a cream with methyl salicylate and Diclofenac in accordance with Example 9.

FIG. 10 shows the composition of a topical analgesic in the form of a cream with borneol in accordance with Example 10.

FIG. 11 shows the composition of a topical analgesic in the form of a cream with borneol in accordance with Example 11.

FIG. 12 shows the composition of a topical analgesic in the form of a cream with borneol in accordance with Example 12.

FIG. 13 shows the composition of a topical analgesic in the form of a gel with borneol in accordance with Example 13.

FIG. 14 shows the composition of a therapeutic massage oil with borneol in accordance with Example 14.

FIG. 15 shows the composition of a topical analgesic in the form of a cream with borneol in accordance with Example 15.

FIG. 16 shows the composition of a therapeutic massage oil with borneol in accordance with Example 16.

FIG. 17 shows the composition of a therapeutic ultrasound gel with borneol in accordance with Example 17.

FIG. 18 shows the composition of a topical analgesic in the form of a cream with methyl salicylate in accordance with Example 18.

FIG. 19 shows the composition of a topical analgesic in the form of a cream without methyl salicylate in accordance with Example 19.

FIG. 20 shows the composition of a therapeutic massage oil in accordance with Example 20.

FIG. 21 shows the composition of a therapeutic ultrasound gel in accordance with Example 21.

FIG. 22 shows the composition of a topical analgesic in the form of a cream with methyl salicylate tit accordance with Example 22.

FIG. 23 shows the composition of a topical analgesic in the form of a cream with cannabidiol in accordance with Example 23.

FIG. 24 shows the composition of a topical analgesic in the form of a cream with 1,8-cineole in accordance with Example 24.

### DESCRIPTION OF THE EMBODIMENTS

Activation of various TRP ion channels cause stimuli such as that the result of chemical, mechanical or thermally induced pain. It is also known that these same TRP ion channels can be inhibited to decrease or eliminate the sensing of pain. Surprisingly, it has been found in this present disclosure that natural compounds can be combined to control gating to inhibit key pain inducing TRP ion channels, including TRPA1 and TRPV1, and to stimulate the TRPM8 ion channel. The control of TRP ion channels is a key embodiment in compositions used in this present disclosure to manufacture topical analgesic compositions and serve as the basis of methods to reduce inflammation and pain. Compositions of this present disclosure can include natural fixed seed oils containing high concentrations of Omega-3 essential fatty acids selected from a group comprising: flaxseed oil, hemp oil, hempseed oil, kiwifruit seed oil, pumpkin seed oil, chia seed oil, algal oil, perilla seed oil and walnut oil.

Compositions in this present disclosure comprise a topical analgesic composition consisting of at least one natural plant extract TRPM8 agonist, at least one natural plant extract TRPA1 antagonist, and optionally at least one fixed plant seed oil TRPV1 antagonists containing Omega-3 fatty acids, and optionally one or more cannabinoid compounds, and optionally methyl salicylate, optionally a NSAID and a carrier. These novel topically administrated compositions and methods relieve inflammation and pain in mammals associated with one or more of nociceptive, neuropathic, somatic pain, radicular pain and associated musculoskeletal, osteoarthritic, muscle, joint, arthritis, rheumatoid arthritis, back, strains and sprains pain associated with sports injuries, post-surgical conditions and other diseases.

Compositions in this present disclosure include menthol, 1,8-cineole and/or borneol and Omega-3 essential fatty acids. These are complementary bioactive natural compounds are used in combination in this present disclosure. Menthol is used as an effective TRPM8 agonist, however in humans it also is a TRPA1 agonist associated with pain and inflammation. Because menthol activates TRPA1, it also has the sensation of cold which can be uncomfortable for human applications. 1,8-cineole is fortuitously another naturally occurring bioactive compound that is a TRPA1 antagonist as well as a TRPM8 agonist. Borneol is fortuitously yet another naturally occurring bioactive compound that is a TRPA1 antagonist as well as a TRPM8 agonist. Borneol and/or 1,8-cineole therefore inhibit pain and inflammation associated with activation of TRPA1 caused by menthol and additionally activates the TRPM8 ion channel. Further, the addition of a TRPA1 antagonist in these topical analgesic formulations reduces the cold sensation in comparison to menthol alone and other menthol containing topical formulations. Further, it has been surprisingly found that the addition of at least one fixed plant seed oil containing high concentrations of Omega-3 essential fatty acids also serves to reduce pain and inflammation by the TRPV1 antagonist mechanism and makes the topical analgesic composition of menthol, 1,8-cineole and/or borneol and/or wintergreen oil less irritating to the skin, as well as facilities transport of the topical pain relief compositions through and into the dermis, epidermis, subcutis and to tissues below the skin.

In one embodiment of this present disclosure is a topical analgesic composition in which the natural plant extract TRPM8 agonist is 1-menthol. In another embodiment of this present disclosure is a composition in which the source of 1-menthol is selected from one or more essential oils selected from the group of: Mentha spp., including, but not limited to Mentha piperita and Mentha arvensis. In a preferred aspect of this present disclosure the topical analgesic composition, in which the natural plant extract TRPM8 agonist is 1-menthol from the essential oil of Mentha arvensis.

In yet another embodiment of this present disclosure is a composition in which the natural plant extract TRPM8 agonist is 1,8-cineole, borneol, linalool, menthone, geraniol, or isopulegol.

In one embodiment of this present disclosure the natural plant extract TRPA1 antagonist is 1,8-cineole from one or more essential oils selected from the group of: Eucalyptus spp., including, but not limited to: Eucalyptus polybractea; Eucalyptus globulus, Eucalyptus radiate, Eucalyptus camaldulensis, Eucalyptus smithii, Rosmarinus spp., including but not limited to; Rosmarinus officinalis; and Salvia spp. including but not limited to Salvia lavandulifolia. In one embodiment of this present disclosure the natural plant extract TRPA1 antagonist is borneol from one or more essential oils selected from the group of: Thymus satureioides (Red Thyme Borneol Type Morrocco) and Cinnamomum burmanni (Mei Pian Tree). In a preferred aspect of this present disclosure the topical analgesic composition in which the natural plant extract TRPA1 antagonist is 1,8-cineole from the essential oil of Eucalyptus globulus. In yet another preferred aspect of this present disclosure the topical analgesic composition in which the natural plant extract TRPA1 antagonist is 1,8-cineole from the essential oil of Rosmarinus officinalis. In yet another preferred aspect of this present disclosure the topical analgesic composition in which the natural plant extract TRPA1 antagonist is borneol from the essential oil of Thymus satureioides. In yet another preferred aspect of this present disclosure the topical analgesic composition in which the natural plant extract TRPA1 antagonist is a mixture of 1,8-cineole from the essential oil of Rosmarinus officinalis and borneol from Thymus satureioides. A person having ordinary skill in the art would recognize that synthetic sources and plant extracts could be used in this present disclosure as sources of menthol, 1,8-cineole, borneol and methyl salicylate instead of natural essential oil plant extracts. A person having ordinary skill in the art would recognize that synthetic sources and plant extracts could be used in this present disclosure as sources of menthol, 1,8-cineole, borneol and methyl salicylate instead of natural essential oils.

In yet another embodiment of this present disclosure cannabinoid (both phytocannabinoid and synthetic cannabinoid) compounds, including but not limited to: 9-Tetrahydrocannabinol (delta-9-THC), 9-THC Propyl Analogue (THC-V), Cannabidiol (CBD), Cannabidiol Propyl Analogue (CBD-V), Cannabinol (CBN), Cannabichromene (CBC), Cannabichromene Propyl Analogue (CBC-V), Cannabigerol (CBG), cannabinoid terpenoids, and cannabinoid flavonoids; cannabinol (CBN) are combined with TRPA1 antagonists and optionally, TRPM8 agonists; and further optionally with one or more NSAIDs and optionally oils high in Omega-3 fatty acids as TRPV1 antagonists. Because of its lack of psychoactive properties CBD is a preferred phytocannabinoid in this disclosure. In a preferred embodiment of this present disclosure is a topical analgesic composition consisting of CBD, a natural plant extract TRPA1 antagonist borneol from the essential oil of Thymus satureioides and flax seed oil high in Omega-3 fatty acids as TRPV1 antagonists. In yet another preferred embodiment of this present disclosure is a topical analgesic composition consisting of CBD, a natural plant extract TRPA1 antagonist borneol from the essential oil of Thymus satureioides, a natural plant extract TRPM8 agonist 1-menthol from the essential oil of Mentha arvensis and flax seed oil high in Omega-3 fatty acids. In yet another preferred embodiment of this present disclosure is a topical analgesic composition consisting of CBD, a natural plant extract TRPA1 antagonist borneol from the essential oil of Thymus satureioides, a natural plant extract TRPA1 antagonist 1,8-cineole from the essential oil of Rosmarinus officinalis, a TRPM8 agonist 1-menthol from the essential oil of Mentha arvensis and flax seed oil high in Omega-3 fatty acids as TRPV1 antagonists.

The compositions of TRPA1 antagonists, TRPM8 agonists and one or more fixed seed oil containing high concentrations of Omega-3 essential fatty acids in this present disclosure comprise therapeutically effective amounts of TRPA1 antagonists TRPM8 agonists and one or more fixed seed oil containing high concentrations of Omega-3 essential fatty acids as TRPV1 antagonists to reduce nociceptive, neuropathic, somatic pain, radicular pain and inflammation associated with musculoskeletal, osteoarthritic, muscle, joint, arthritis, rheumatoid arthritis, back, strains and sprains pain associated with sports injuries and other diseases or trauma. The compositions can be applied, for example once, twice, or even four times per day to skin that is unbroken and not bleeding.

In one embodiment of the present disclosure, the compositions and methods to reduce pain and inflammation optionally comprise methyl salicylate, a bioactive compound that undergoes biotransformation in mammals to salicylic acid following absorption through the skin. Methyl salicylate therefore is a prodrug to salicylic acid, a known COX-2 inhibitor. COX-2 inhibitors prevent inflammation and pain and in one embodiment of this present disclosure are unexpectedly effective as a result of the synergy of methyl salicylate in combination with TRPA1 antagonists and TRPM8 agonists. The addition of 1,8-cineole and/or borneol in this present disclosure also is effective at inactivating the gating of the TRPA1 ion channel associated with methyl salicylate.

In one embodiment of this present disclosure is a topical analgesic composition further comprising the addition of methyl salicylate as a COX-2 inhibitor with TRPA1 antagonists, TRPM8 agonists and one or more fixed seed oil containing high concentrations of Omega-3 essential fatty acids as TRPV1 antagonists.

In a preferred aspect of this present disclosure the topical analgesic composition in which methyl salicylate is from a natural essential oil source from Gaultheria procumbens.

A person having ordinary skill in the art would recognize that synthetic sources could be used in this present disclosure as sources of methyl salicylate instead of natural essential oil plant extracts.

The compositions of methyl salicylate, TRPA1 antagonists, TRPM8 agonists and one or more fixed seed oil containing high concentrations of Omega-3 essential fatty acids as TRPV1 antagonists in this present disclosure comprise therapeutically effective amounts of methyl salicylate. TRPA1 antagonists, TRPM8 agonists and one or more fixed seed oil containing high concentrations of Omega-3 essential fatty acids to reduce nociceptive, neuropathic, somatic pain, radicular pain and inflammation associated with musculoskeletal, osteoarthritic, muscle, joint, arthritis, rheumatoid arthritis, back, strains and sprains pain associated with sports injuries and other diseases or trauma. The compositions can be applied, for example once, twice, or even four times per day to skin that is unbroken and not bleeding.

The compositions of cannabinoid compounds, TRPA1 antagonists, TRPM8 agonists and one or more fixed seed oil containing high concentrations of Omega-3 essential fatty acids in this present disclosure comprise therapeutically effective amounts of cannabinoid compounds, TRPA1 antagonists, TRPM8 agonists and one or more fixed seed oil containing high concentrations of Omega-3 essential fatty acids as TRPV1 antagonists to reduce nociceptive, neuropathic, somatic pain, radicular pain and inflammation associated with musculoskeletal, osteoarthritic, muscle, joint, arthritis, rheumatoid arthritis, back, strains and sprains pain associated with sports injuries and other diseases or trauma. The compositions can be applied, for example once, twice, or even four times per day to skin that is unbroken and not bleeding.

In yet another embodiment of this present disclosure, compositions and methods to reduce pain and inflammation additionally comprise a topically active NSAID, for example but not limited to diclofenac or a salt of diclofenac thereof. Compositions of NSAIDs in combination with the TRPA1 antagonists, TRPM8 agonists and one or more fixed seed oils containing high concentrations of Omega-3 essential fatty acids as TRPV1 antagonists in this present disclosure are unexpectedly effective in pain relief and reduction of inflammation and methods to treat pain and inflammation. In yet another embodiment of this present disclosure methyl salicylate and a NSAID are combined with the TRPA1 antagonists, TRPM8 agonists and one or more fixed seed oil containing high concentrations of Omega-3 essential fatty acids as TRPV1 antagonists in this present disclosure and are unexpectedly effective in pain relief and reduction of inflammation and methods to treat pain and inflammation. The NSAID is selected from the group of arthrotec, celecoxib, rofecoxib, vadecoxib, naproxen, ketoprofen, felbinac, ibuprofen, piroxicam, benzydamine, indomethacin and diclofenac. Included in the NSAID definition used herein are pharmaceutically active salts of the forgoing group.

In a preferred aspect of this present disclosure the NSAID in a topical analgesic composition of this present disclosure is diclofenac.

Accordingly, this present disclosure provides topical analgesic compositions that are administered for the treatment of pain and inflammation associated with nociceptive, neuropathic, somatic pain, radicular pain and methods for reducing such pain in mammals. These compositions can be applied, for example once, twice, or even four times per day to skin that is unbroken and not bleeding.

The pharmaceutical compositions of a topically active NSAID and optionally methyl salicylate combined with the TRPA1 antagonists, TRPM8 agonists and one or more fixed seed oil containing high concentrations of Omega-3 essential fatty acids as TRPV1 antagonists in this present disclosure comprise a therapeutically effective amount of a NSAID and of methyl salicylate to reduce nociceptive, neuropathic, somatic pain, radicular pain and inflammation associated with musculoskeletal, osteoarthritic, muscle, joint, arthritis, rheumatoid arthritis, back, strains and sprains pain associated with sports injuries and other diseases or trauma. The compositions can be applied, for example once, twice, or even four times per day to skin that is unbroken and not bleeding.

A topically active NSAID is meant as a NSAID when used in combination with a suitable carrier can be transported through the skin barrier of mammals and becomes locally active in and below the skin and is safe for exposure to skin without unacceptable reactions.

Yet another aspect of this present disclosure is the incorporation of the composition into either a hydrophilic or hydrophobic base for use as a sprayable liquid, a gel, a massage oil, an ointment, a cream, a stick or a patch.

In one embodiment of the present disclosure the composition comprises a topical analgesic wherein a major portion by weight of said carrier is a hydrophilic alcohol. In a preferred aspect of this present disclosure the hydrophilic alcohol is isopropyl alcohol. The sprayable liquid may be applied from a hand-pumped spray bottle or alternatively, an aerosol from an inert gas pressurized container spray.

In one embodiment of the present disclosure the composition comprises a topical analgesic wherein the carrier forms a viscous gel consisting of at least one thickening agent and water. A thickening agent can be selected from a carbomer, cacia, alginic acid, bentonite, carboxymethyl cellulose, ethylcellulose, hydroxyethyl cellulose, hydroxypropyl cellulose, magnesium aluminum silicate (Veegum), methylcellulose, poloxamers (Pluronics), polyvinyl alcohol, sodium alginate, tragacanth, guar gum, and xanthan gum. In a preferred aspect of this present disclosure the preferred thickener is the carbomer sodium polyacrylate. In one aspect of this present disclosure, the carrier is a viscous gel composition wherein the thickening agent is mixed with the oil phase, consisting of one or more of the hydrophobic components of the composition, then mixed with water.

In another embodiment of the present disclosure the composition comprises a topical analgesic wherein the carrier forms a viscous cream consisting of at least one thickening agent, a surfactant and water. In one aspect of this disclosure the surfactant comprises a non-ionic surfactant. The non-ionic surfactant can be selected from a group comprising: polyoxyethylene (20) sorbitan monolaurate; polyoxyethylene (20) sorbitan monooleate; polyoxyethylene (20) sorbitan monopalmitate; polyoxyethylene (20) sorbitan monostearate; sorbitan trioctadecanoate; polyglyceryl-3 Stearate; polyglyceryl-3 palmitate; polyglyceryl-2 laurate; polyglyceryl-5 laurate; polyglyceryl-5 oleate; polyglyceryl-5 dioleate; and polyglyceryl-10 diisostearate. In a preferred embodiment of this present disclosure the viscous cream is comprised of the surfactant polyoxyethylene (20) sorbitan monolaurate, the thickening agent sodium polyacrylate and water. In one aspect of this present disclosure, the carrier is a viscous cream-like gel composition wherein the thickening agent and the surfactant are mixed with the oil phase, consisting of one or more of the hydrophobic components of the composition, then mixed with water. For the purpose of this disclosure the gel is transparent and the cream is not transparent to light.

Yet another embodiment of the present disclosure the composition comprises a hydrophobic topical analgesic wherein the carrier forms a wax consisting of at least one wax and optionally, at least one oil. In one aspect of the present disclosure wax is selected from beeswax, candelilla wax, carnauba wax and jojoba wax and the fixed seed oil is one or more of flaxseed oil, hemp oil, kiwifruit seed oil, pumpkin seed oil, chia seed oil, algal oil, perilla seed oil and walnut oil. In a preferred aspect of this present disclosure the preferred wax composition consists of beeswax at a concentration from about 25 to about 98% by weight and flax seed oil at a concentration from about 2 to about 75% by weight.

Yet in another embodiment of the present disclosure is a composition comprising a hydrophobic topical analgesic in which the carrier is a fixed plant oil or seed oil or a mixture of fixed plant and/or seed oils to form a therapeutic massage oil. In one embodiment of the present disclosure the fixed plant and seed oils is one or more of sweet almond oil flax seed oil, evening primrose oil, jojoba oil, apricot kernel oil, grape seed oil, hemp seed oil, chia seed oil, algal oil, perilla seed oil hemp oil. In a preferred embodiment of this present disclosure the fixed plant oil and seed oil composition comprises sweet almond oil (29.89%), grape seed oil (40.11%), apricot kernel oil (13.04%), hemp seed oil (10.87%), evening primrose oil (2.72%) and jojoba oil (2.72%) and the TRPA1 antagonists are rosemary essential oil (0.27%) and divine essential oil (0.27%), the TRPM8 agonist is peppermint essential oil (0.27%) and the COX-2 inhibitor is wintergreen essential oil (0.27%). The pH of healthy skin is about 4.7. The pH of the compositions in this present disclosure can be from pH of about 4.5 up to a pH of about 7.3. Activation of TRPM8 does not appear to be affected over this pH range.

Preferred embodiments of this disclosure are described in clauses 1-117 below.

1. A topical analgesic composition comprising at least one natural plant extract TRPM8 agonist, at least one natural plant extract TRPA1 antagonist, at least one fixed plant seed oil containing TRPV1 antagonist Omega-3 fatty acids, and optionally a carrier.

2. The topical analgesic composition of clause 1 further comprising a nonsteroidal anti-inflammatory agent (NSAID).

3. The topical analgesic composition of clause 2 where the NSAID agent is selected from the group consisting of arthrotec, celecoxib, rofecoxib, vadecoxib, naproxen, ketoprofen, felbinac, ibuprofen, piroxicam, benzydamine, indomethacin and diclofenac.

4. The topical analgesic composition of clause 3 where the NSAID is diclofenac.

5. The topical analgesic composition of clause 1 further comprising methyl salicylate.

6. The topical analgesic composition of clause 5 in which the source of the methyl salicylate is an essential oil from Gaultheria procumbens (wintergreen).

7. The topical analgesic composition of clause 1 in which the natural plant extract TRPM8 agonist is 1-menthol.

8. The topical analgesic composition of clause 7 in which 1-menthol is from a synthetic source or is derived from one or more essential oils selected from the group consisting of: Mentha spp., Mentha piperita, and Mentha arvensis.

9. The topical analgesic composition of clause 1 in which the natural plant extract TRPM8 agonist is menthone, 1,8-cineole, borneol, linalool, geraniol, or isopulegol.

10. The topical analgesic composition of clause 1 in which the natural plant extract TRPA1 antagonist is 1,8-cineole from a synthetic source or derived from one or more essential oils selected from the group consisting of Eucalyptus spp. including Eucalyptus polybractea, Eucalyptus globulus, Eucalyptus radiate, Eucalyptus camaldulensis, Eucalyptus smithii, and Eucalyptus globulus; and Rosmarinus spp., including Rosmarinus officinalis; and Salvia lavandulifolia.

11. The topical analgesic composition of clause 1 in which the natural plant extract TRPA1 antagonist is borneol from a synthetic source or derived from one or more of the essential oils selected from the group consisting of: Thymus satureioides and Cinnamomum burmanni.

12. The topical analgesic composition of clause 11 in which the TRPA1 antagonist is borneol derived from the essential oil of Thymus satureioides.

13. The topical analgesic composition of clause 1 further comprising one or more cannabinoid compounds.

14. The topical analgesic composition of clause 13 in which the one or more cannabinoid compounds include cannabidiol.

15. The topical analgesic composition of clause 13 in which the one or more cannabinoid compounds include cannabidivarin.

16. The topical analgesic composition of clause 13 in which the one or more cannabinoid compounds further contains delta 9-tetrahydrocannabinol.

17. The topical analgesic composition of clause 1 in which the fixed plant seed oil containing TRPV1 antagonist Omega-3 fatty acids is selected from a group consisting of: flaxseed oil, hemp oil, hemp seed oil, kiwifruit seed oil, olive oil, pumpkin seed oil, chia seed oil, algal oil, perilla seed oil and walnut oil.

18. The topical analgesic composition of clause 1 wherein a major portion by weight of said carrier is a hydrophilic alcohol.

19. The topical analgesic composition of clause 18 in wherein the hydrophilic alcohol is isopropyl alcohol.

20. The topical analgesic composition of clause 1 wherein the carrier is comprised of water and a thickening agent to form a viscous gel.

21. The topical analgesic composition of clause 20 further comprising a non-ionic surfactant to form a viscous cream.

22. The topical analgesic composition of clause 21 wherein the surfactant is selected from a group comprising: polyoxyethylene (20) sorbitan monolaurate, polyoxyethylene (20) sorbitan monooleate; polyoxyethylene (20) sorbitan monopalmitate; polyoxyethylene (20) sorbitan monostearate; sorbitan trioctadecanoate; polyglyceryl-3 stearate; polyglyceryl-3 palmitate; polyglyceryl-2 laurate; polyglyceryl-5 laurate; polyglyceryl-5 oleate; polyglyceryl-5 dioleate; and polyglyceryl-10 diisostearate.

23. The topical analgesic composition of clause 20 wherein the thickening agent is selected from the group consisting of: a carbomer, cacia, alginic acid, bentonite, carboxymethyl cellulose, ethylcellulose, hydroxyethyl cellulose, hydroxypropyl cellulose, magnesium aluminum silicate (Veegum), methylcellulose, poloxamers (Pluronics), polyvinyl alcohol, sodium alginate, tragacanth, guar gum, and xanthan gum.

24. The topical analgesic composition of clause 20 where the thickener is carbomer sodium polyacrylate.

25. The topical analgesic composition of clause 1 in which the carrier is comprised of a wax and optionally a fixed oil.

26. The topical analgesic composition of clause 25 wherein the wax is selected from the group consisting of beeswax, candelilla wax, carnauba wax and jojoba wax, and the fixed oil is one or more of apricot kernel oil, borage oil, castor oil, cocoa butter, coconut oil, hemp seed oil, flaxseed oil, kiwifruit seed oil, olive oil, pumpkin seed oil, sweet almond oil, tamanu oil, chia seed oil, algal oil, perilla seed oil and walnut oil.

27. A topical analgesic composition comprising:
a) from about 0.5 to about 10% by weight of at least one TRPM8 agonist;
b) from about 0.5 to about 10% by weight of at least TRPA1 antagonist;
c) optionally from about 0.01 to about 1.000% by weight of at least one NSAID;
d) optionally from about 1 to about 10% by weight of a methyl salicylate;
e) from about 1 to about 10% by weight of at least one fixed plant seed oil containing TRPV1 antagonist Omega-3 fatty acids;
f) optionally from 0.1 to about 1.0% of one or more cannabinoid compounds; and
g) optionally a carrier.

28. The topical analgesic composition as in clause 27 wherein:
component (a) comprises 1-menthol;
component (b) comprises 1,8-cineole;
component (c) comprises diclofenac;
component (d) comprises methyl salicylate;
component (e) comprises flax seed oil;
component (f) comprises cannabidiol.

29. The topical analgesic composition as in clause 27 in which the carrier is a gel comprising of at least one thickener and water.

30. The topical analgesic composition in clause 22 in which the thickener is sodium polyacrylate at a concentration from 1 to 50 g/kg.

31. The topical analgesic composition as in clause 27 in which the carrier is a cream comprising at least one thickener, at least one non-ionic surfactant, and water.

32. The topical analgesic composition as is clause 31 in which the thickener is sodium polyacrylate at a concentration from 1 to 50 g/kg, the non-ionic surfactant polyoxyethylene (20) sorbitan monolaurate at a concentration from 0.5 to 25 g/kg, and water at a concentration from about 80 to 97 percent by weight.

33. The topical analgesic composition of as in clause 27 wherein the carrier comprises a sprayable or aerosol mixture of isopropyl alcohol and optionally water.

34. The topical analgesic composition of clause 33 is which the concentration of isopropyl alcohol is from about 50 to about 97% by weight, and the concentration of water is from about 2.5 to about 50% by weight.

35. The topical analgesic composition of clause 27 in which the carrier is a wax and optionally a fixed oil.

36. The topical analgesic composition of clause 35 in which the wax is one or more of beeswax, candelilla wax, carnauba wax and jojoba wax, and the fixed seed oil is one or more of apricot kernel oil, borage oil, castor oil, cocoa butter, coconut oil, hemp oil, hemp seed oil, flaxseed oil, kiwifruit seed oil, olive oil, pumpkin seed oil, sweet almond oil, tamanu oil, chia seed oil, algal oil, perilla seed oil and walnut oil.

37. The topical analgesic composition of clause 36 in which the wax is beeswax at a concentration from about 25 to about 98% by weight and the fixed oil is flax seed oil at a concentration from about 2 to about 60% by weight.

38. The topical analgesic composition of clauses 29, 31, 33 and 35 absorbed onto natural or synthetic fibers to form a patch.

39. A method of relieving pains in mammals from one or more of nociceptive, neuropathic, somatic pain, radicular pain and associated musculoskeletal, osteoarthritic, muscle, joint, arthritis, rheumatoid arthritis, back, strains and sprains pain associated with sports and other diseases or trauma, by topically administering a composition comprising at least one natural plant extract TRPM8 agonist, at least one natural plant extract is a TRPA1 antagonist, at least one fixed plant seed oil containing TRPV1 antagonist Omega-3 fatty acids, and optionally a carrier.

40. The method of clause 39 further comprising a NSAID.

41. The method of clause 40 where the NSAID is selected from the group consisting of: arthrotec, celecoxib, rofecoxib, vadecoxib, naproxen, ketoprofen, felbinac, ibuprofen, piroxicam, benzydamine, indomethacin and diclofenac.

42. The method of clause 40 where the NSAID is dichlofenac.

43. The method of clause 39 further comprising methyl salicylate.

44. The method of clause 43 where the methyl salicylate is from an essential oil derived from Gaultheria procumbens (wintergreen).

45. The method of clause 39 in which the natural plant extract TRPM8 agonist is 1-menthol.

46. The method of clause 45 in which the source of 1-menthol is from a synthetic source or is selected from one or more essential oils selected from the group of: Mentha spp., including, but not limited to; Mentha Piperita; and Mentha arvensis.

47. The method of clause 19 in which the natural plant extract TRPM8 agonist is menthone, 1,8-cineole, borneol, linalool, geraniol, or isopulegol.

48. The method of clause 39 in which the natural plant extract TRPA1 antagonist is 1,8-cineole from a synthetic source or derived from one or more essential oils selected from the group consisting of: Eucalyptus spp., including Eucalyptus polybractea, Eucalyptus globulus, Eucalyptus radiate, Eucalyptus camaldulensis, Eucalyptus smithii, and Eucalyptus globulus; and Rosmarinus spp., including Rosmarinus officinalis; and Salvia lavandulifolia.

49. The method of clause 39 in which the natural plant extract TRPA1 antagonist is borneol from a synthetic source or derived from one or more of the essential oils selected from the group consisting of: Thymus satureioides and Cinnamomum burmanni.

50. The method of clause 49 in which the TRPA1 antagonist is borneol derived from the essential oil of Thymus satureioides.

51. The method of clause 39 further comprising one or more cannabinoid compounds.

52. The method of clause 51 in which the one or more cannabinoid compounds include cannabidiol.

53. The method of clause 51 in which the one or more cannabinoid compounds include cannabidivarin

54. The method of clause 51 in which the one or more cannabinoid compounds further contain delta9-tetrahydrocannabinol.

55. The method of clause 39 in which the fixed plant seed oil containing TRPV1 antagonist Omega-3 fatty acids is selected from the group consisting of: flaxseed oil, hemp oil, hemp seed oil, kiwifruit seed oil, pumpkin seed oil, chia seed oil, algal oil, perilla seed oil and walnut oil.

56. The method of clause 39 wherein the composition is in a sprayable carrier comprising a hydrophilic alcohol and optionally water.

57. The method of clause 56 wherein the hydrophilic alcohol is isopropyl alcohol.

58. The method of clause 39 wherein the carrier is comprised of water and a thickening agent.

59. The method of clause 58 wherein the thickening agent is selected from the group consisting of: a carbomer, cacia, alginic acid, bentonite, carboxymethyl cellulose, ethylcellulose, hydroxyethyl cellulose, hydroxypropyl cellulose, magnesium aluminum silicate (Veegum), methylcellulose, poloxamers (Pluronics) polyvinyl alcohol, sodium alginate, tragacanth, guar gum, and xanthan gum.

60. The method of clause 59 wherein the thickening agent is a sodium polyacrylate carbomer to form a gel.

61. The method of clause 58 where thickening agent composition further comprising a non-ionic surfactant to form a cream.

62. The method of clause 61 wherein the non-ionic surfactant is selected from the group consisting of: polyoxyethylene (20) sorbitan monolaurate; polyoxyethylene (20) sorbitan monooleate; polyoxyethylene (20) sorbitan monopalmitate; polyoxyethylene (20) sorbitan monostearate; sorbitan trioctadecanoate; polyglyceryl-3 stearate; polyglyceryl-3 palmitate; polyglyceryl-2 laurate; polyglyceryl-5 laurate; polyglyceryl-5 oleate; polyglyceryl-5 dioleate; and polyglyceryl-10 diisostearate.

63. The method of clause 39 in which the carrier is a wax and optionally a fixed oil.

64. The method of clause 63 in which the wax is one or more of beeswax, candelilla wax, carnauba wax and jojoba wax, and the fixed oil is one or more of apricot kernel oil, borage oil, castor oil, cocoa butter, coconut oil, hemp oil, hemp seed oil, flaxseed oil, kiwifruit seed oil, olive oil, pumpkin seed oil, sweet almond oil, tamanu oil, chia seed oil, algal oil, perilla seed oil and walnut oil.

65. A method of relieving pains in mammals from one or more of nociceptive, neuropathic, somatic pain, radicular pain and associated musculoskeletal, osteoarthritic, muscle, joint, arthritis, rheumatoid arthritis, back, strains and sprains pain associated with sports injuries and other diseases or trauma, by topically administering a composition comprising:
a) from about 0.5 to about 10% by weight of at least one TRPM8 agonist;
b) from about 0.5 to about 10% by weight of at least TRPA1 antagonist;
c) optionally from about 0.01 to about 1.000% by weight of at least one NSAID;
d) optionally from about 1 to about 10% by weight of a methyl salicylate;
e) from about 1 to about 10% by weight of at least one fixed plant seed oil containing TRPV1 antagonist Omega-3 fatty acids;
f) optionally from 0.1 to about 1.0% of one or more cannabinoid compounds; and
g) optionally a carrier.

66. The method as in clause 65 wherein:
component (a) comprises 1-menthol;
component (b) comprises 1,8-cineole;
component (c) comprises diclofenac;
component (d) comprises methyl salicylate;
component (e) comprises flax seed oil; and
component (f) comprises cannabidiol.

67. The method as in clause 65 in which the carrier is a gel comprising at least one thickener and water.

68. The method as in clause 67 in which the thickener is sodium polyacrylate at a concentration from 1 to 50 g/kg.

69. The method as in clause 65 in which the carrier is a cream comprising at least one thickener, at least one non-ionic surfactant, and water.

70. The method as in clause 65 in which the thickener is sodium polyacrylate polyacrylate at a concentration from 1 to 50 g/kg, the non-ionic surfactant polyoxyethylene (20) sorbitan monolaurate at a concentration from 0.5 to 25 g/kg, and water at a concentration from about 80 to 98 percent by weight.

71. The method as in clause 65 wherein the carrier comprises a sprayable or aerosol mixture of isopropyl alcohol and optionally water.

72. The method as in clause 71 is which the concentration of isopropyl alcohol is from about 50 to about 97% by weight, and the concentration water is from about 2.5 to 50% by weight.

73. The method as in clause 65 which the carrier is a wax and optionally a fixed oil.

74. The method as is clause 65 in which the wax is one or more of beeswax, candelilla wax, carnauba wax and jojoba wax, and the fixed plant seed oil is one or more of apricot kernel oil, borage oil, castor oil, cocoa butter, coconut oil, hemp oil, hemp seed oil, flaxseed oil, kiwifruit seed oil, olive oil, pumpkin seed oil, sweet almond oil, tamanu oil, chia seed oil, algal oil, perilla seed oil and walnut oil.

75. The method as in clause 74 in which the wax is beeswax at a concentration from about 25 to about 98% by weight, and the fixed plant seed oil is flax seed oil at a concentration from about 2 to about 60% by weight.

76. The method as in clauses 67, 69, 71 and 73 wherein the composition is absorbed onto natural or synthetic fibers to form a patch.

77. An analgesic composition comprising at least one TRPM8 agonist, TRPA1 antagonist, and a nonsteroidal anti-inflammatory agent (NSAID).

78. The analgesic composition of clause 77 where the NSAID agent is selected from the group consisting of arthrotec, celecoxib, rofecoxib, vadecoxib, naproxen, ketoprofen, felbinac, ibuprofen, piroxicam, benzydamine, indomethacin and diclofenac.

79. The analgesic composition of clause 77 where the NSAID is diclofenac.

80. The analgesic composition of clause 77 in which the TRPM8 agonist is 1-menthol.

81. The analgesic composition of clause 80 in which 1-menthol is from a synthetic source or is derived from one or more essential oils selected from the group consisting of: Mentha spp., including Mentha piperita; and Mentha arvensis.

82. The analgesic composition of clause 77 in which the TRPM8 agonist is menthone, 1,8-cineole, borneol, linalool, geraniol, or isopulegol.

83. The analgesic composition of clause 77 in which the TRPA1 antagonist is a synthetic compound.

84. The analgesic composition of clause 77 in which the TRPA1 antagonist is 1,8-cineole from a synthetic source or derived from natural sources comprising one or more essential oils selected from the group consisting of: Eucalyptus spp., including Eucalyptus polybractea, Eucalyptus globulus, Eucalyptus radiate, Eucalyptus camaldulensis, Eucalyptus smithii, and Eucalyptus globulus; Rosmarinus spp., including Rosmarinus officinalis; and Salvia lavandulifolia.

85. The analgesic composition of clause 77 in which the TRPA1 antagonist is borneol from a synthetic source or derived from one or more of the essential oils selected from the group of: Thymus satureioides and Cinnamomum burmanni.

86. The analgesic composition of clause 77 in which the TRPA1 antagonist is borneol derived from the essential oil of Thymus satureioides.

87. An analgesic composition comprising at least one TRPM8 agonist, TRPA1 antagonist, and one or more natural or synthetically derived cannabinoid compounds.

88. The analgesic composition of clause 87 in which the cannabinoid compound includes cannabidiol.

89. The analgesic composition of clause 87 in which the cannabinoid compound includes cannabidivarin.

90. The analgesic composition of clause 87 in which the cannabinoid compounds are derived from hemp oil.

91. The analgesic composition of clause 87 in which the cannabinoid compound is cannabidiol derived from hemp oil.

92. The analgesic composition of clause 87 in which the cannabinoid compound is delta-9-tetrahydrocannabinbol derived from hemp oil.

93. The analgesic composition of clause 87 in which the composition further comprises a carrier.

94. The analgesic composition of clause 93 in wherein carrier can be a paste, a liquid, a gel, a wax or a cream.

95. A method of relieving pains in mammals by administering an analgesic composition comprising of at least one TRPM8 agonist, at least one TRPA1 antagonist, and a nonsteroidal anti-inflammatory agent (NSAID).

96. The method of clause 95 where the NSAID agent is selected from the group of arthrotec, celecoxib, rofecoxib, vadecoxib, naproxen, ketoprofen, felbinac, ibuprofen, piroxicam, benzydamine, indomethacin and diclofenac.

97. The method of clause 95 where the NSAID is diclofenac.

98. The method of clause 95 where the TRPM8 agonist is a synthetic compound.

99. The method of clause 95 in which the TRPM8 agonist is 1-menthol.

100. The method of clause 99 in which the source of 1-menthol is selected from one or more essential oils selected from the group of: Mentha spp., including, but not limited to, Mentha piperita, and Mentha arvensis.

101. The method of clause 95 in which the TRPM8 agonist is menthone, 1,8-cineole, borneol, linalool, geraniol or isopulegol.

102. The method of clause 95 in which the TRPA1 antagonist is a synthetic compound.

103. The method of clause 95 in which the TRPA1 antagonist is 1,8-cineole from a synthetic source or derived from natural sources comprising one or more essential oils selected from the group of: Eucalyptus spp., including Eucalyptus polybractea; Eucalyptus globulus, Eucalyptus radiate, Eucalyptus camaldulensis, Eucalyptus smithii, and Eucalyptus globulus; Rosmarinus spp. including Rosmarinus officinalis; and Salvia lavandulifolia.

104. The method of clause 95 in which, the TRPA1 antagonist is borneol derived from one or more of the essential oils selected from the group of: Thymus satureioides and Cinnamomum burmanni.

105. The method of clause 95 in which the TRPA1 antagonist is borneol derived from the essential oil of Thymus satureioides.

106. The method of clause 95 wherein the composition is administered orally or topically.

107. A method of relieving pains in mammals by administering an analgesic composition comprising of at least one TRPM8 agonist, at least one TRPA1 antagonist, and at least one natural or synthetically derived cannabinoid compound.

108. The method of clause 107 in which the cannabinoid compound includes cannabidiol.

109. The method of clause 107 in which the cannabinoid compound includes cannabidivarin.

110. The method of clause 107 in which the cannabinoid compounds are derived from hemp oil.

111. The method of clause 107 in which the cannabinoid compound is cannabidiol derived from hemp oil.

112. The method of clause 107 in which the cannabinoid compound is delta9-tetrahydrocannabinbol derived from hemp oil.

113. The method of clause 107 where carrier comprises a paste, a liquid, a gel, a wax or a cream.

114. The method of clause 107 wherein the composition is administered orally or topically.

115. The topical analgesic composition of clause 1 or the analgesic composition of clause 77 which is incorporated into either a hydrophilic or hydrophobic base, suspensions, solids, semi-solids, powders, or nanoparticles for a pharmaceutical compositions, in which the pharmaceutical composition comprises one or more of a sprayable liquid, a gel, a lotion, a film, an ointment, a massage oil, a cream, a paste, a stick, a patch, tablets, capsules, powders or granules.

116. The manufacture of a pharmaceutical composition including the topical analgesic composition of clause 1 or the analgesic composition of clause 77 in which the pharmaceutical composition comprises a solid, semi-solid, powder or granule with one or more excipients selected from the group consisting of diluents, fillers, binders, adhesives, disintegrants, lubricants, anti-adhesives glidents, coloring agents, sweeteners, coating agents, plasticizers, wetting agents, buffers lactose, dibasic calcium phosphate, sucrose, corn (maize) starch, microcrystalline cellulose or modified cellulose including hydroxypropyl methylcellulose and hydroxyethylcellulose.

117. The topical analgesic composition of clause 1 or the analgesic composition of clauses 77 and 93 in which the carrier is selected from the group consisting of a paste, a liquid, a gel, a wax, a cream, a suspension, a film, a stick, a patch, a solid, a powder, nanoparticles, and granules; and in which the carrier comprises one or more additives or excipients selected from the group consisting of odorants, deodorants, diluents, fillers, binders, adhesives, disintegrants, lubricants, anti-adhesives glidents, coloring agents, sweeteners, coating agents, plasticizers, wetting agents, and buffers.

The following examples illustrate the present disclosure but are not intended to limit scope of the compositions, the methods of manufacture and use of the topical analgesics as described above. Temperature are given in degrees centigrade (° C.) and unless otherwise noted all temperatures are at 25° C.

### EXAMPLE 1

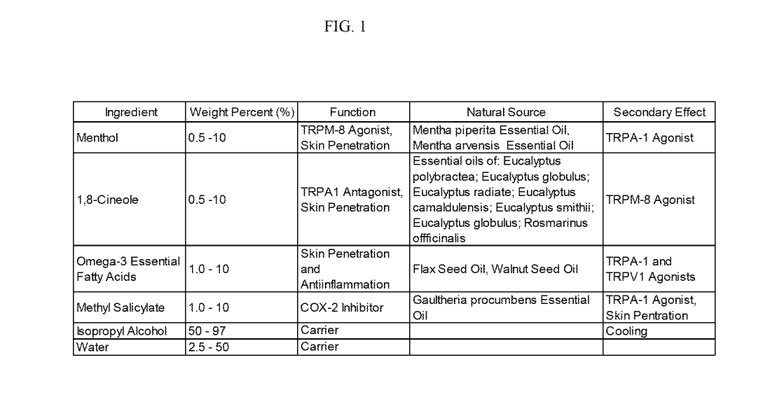

A method of manufacture of the topical analgesic sprayable liquid containing methyl salicylate, 1,8-cineole, menthol and Omega-3 fatty acids for the composition provided in Example 1 consists of mixing an amount of water with isopropyl alcohol with the other ingredients in a ratio of alcohol to water, such that a stable single phase homogeneous solution results. Mixing is limited to that required to create a stable single phase homogeneous solution and to minimize volatilization of menthol and 1,8-cineole. Methods of use of the composition of the topical analgesic given in Example 1 include but are not meant to be limited to placing the composition in a spray bottle and spraying onto the skin, placing the composition in a closed aerosol spray vessel under pressure of an inert gas and spraying on the skin and wetting a patch a placing on the skin. The topical analgesic sprayable liquid composition is Example 1 can optionally be made with borneol or a mixture of 1,8-cineole and borneol in the same total concentration range as 1,8-cineole alone presented in Example 1. The composition of a topical analgesic in the form of a sprayable liquid or aerosol with methyl salicylate is shown in FIG. 1.

### EXAMPLE 2

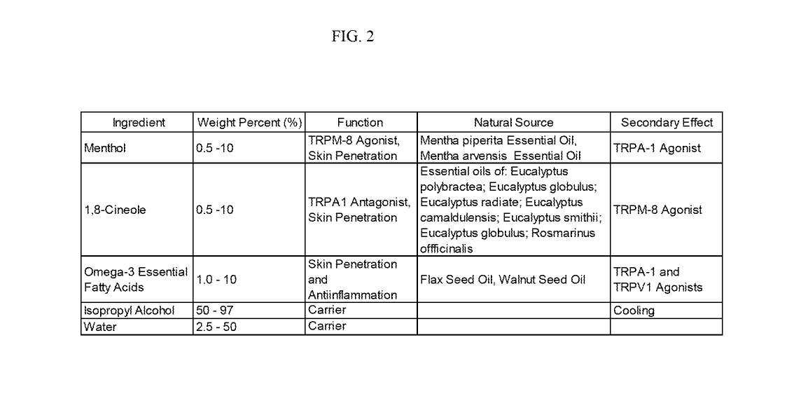

A method of manufacture of the topical analgesic sprayable liquid containing 1,8-cineole, menthol and Omega-3 fatty acids for the composition presented in Example 2 consists of mixing an amount of water with isopropyl alcohol with the other ingredients in a ratio of alcohol to water, such that a stable single phase homogeneous solution results. Mixing is limited to that required to create a stable single phase homogeneous solution and to minimize volatilization of menthol and 1,8-cineole. Methods of use of the composition of the topical analgesic given in Example 2 include but are not meant to be limited to placing the composition in a spray bottle and spraying onto the skin, placing the composition in a closed aerosol spray vessel under pressure of an inert gas and spraying on the skin and wetting and patch a placing on the skin. The topical analgesic sprayable liquid composition in Example 2 can optionally be made with borneol or a mixture of 1,8-cineole and borneol in the same total concentration range as 1,8-cineole alone presented in Example 2. The composition of a topical analgesic in the form of a sprayable liquid or aerosol without methyl salicylate is shown in FIG. 2.

### EXAMPLE 3

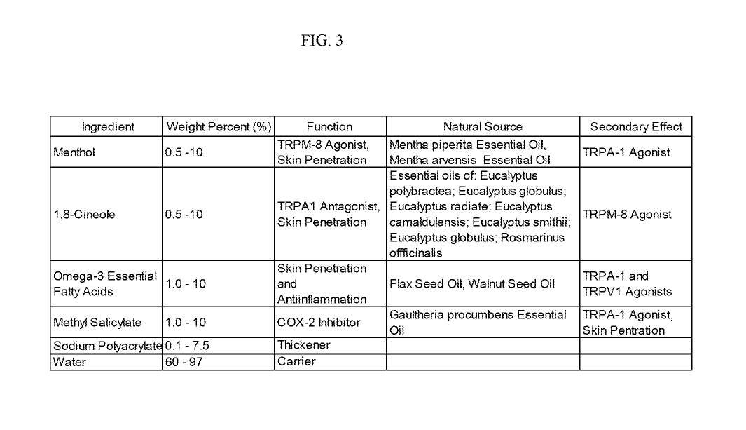

A method of manufacture of the topical analgesic gel containing methyl salicylate, 1,8-cineole, menthol Omega-3 fatty acids for the composition presented in Example 3 consists of mixing an amount sodium polyacrylate with the oil phase components of the compositing to dissolve the sodium polyacrylate then adding an amount of water, such that a stable single phase homogeneous gel results. Mixing is limited to that required to dissolve he sodium polyacrylate with the oil phase components and then to mix the water for a period of time to create the stable single phase homogeneous gel and to minimize volatilization of menthol and 1,8-cineole. Methods of use of the composition of the topical analgesic given in Example 3 include but are not meant to be limited to placing the gel composition in a roll-on bottle then placing the roll-on ball into the roll-on bottle container, placing the gel composition in a squeeze tube container, placing the gel composition in a hand pump bottle container and applying a therapeutic amount of the gel on the skin and wetting a patch with the gel and placing on the skin. The topical analgesic gel composition in Example 3 can optionally be made with borneol or a mixture of 1,8-cineole and borneol in the same total concentration range as 1,8-cineole alone presented in Example 3. The composition of a topical analgesic in the form of a gel with methyl salicylate is shown in FIG. 3.

### EXAMPLE 4

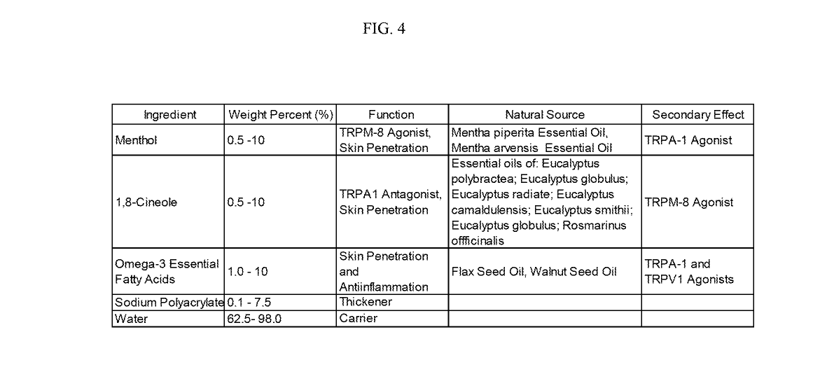

A method of manufacture of the topical analgesic gel containing 1,8-cineole menthol and Omega-3 fatty acids and not containing methyl salicylate for the composition presented in Example 4 consists of mixing an amount sodium polyacrylate with the oil phase components (1,8-cineole, menthol and Omega-3 fatty acids) of the compositing to dissolve the sodium polyacrylate then adding an amount of water, such that a stable single phase homogeneous gel results. Mixing is limited to that required to dissolve the sodium polyacrylate with the oil phase components and then to mix the water for a period of time to create the stable single phase homogeneous gel and to minimize volatilization of menthol and 1,8-cineole. Methods of use of the composition of the topical analgesic given in Example 4 include but are not meant to be limited to placing the gel composition in a roll-on bottle then placing the roll-on ball into the roll-on bottle container, placing the gel composition in a squeeze tube container placing the gel composition in a hand pump bottle container and applying a therapeutic amount of the gel on the skin and wetting a patch with the gel and placing on the skin. The topical analgesic gel composition in Example 4 can optionally be made with borneol or a mixture of 1,8-cineole and borneol in the same total concentration range as 1,8-cineole alone presented in Example 4. The composition of a topical analgesic in the form of a gel without methyl salicylate is shown in FIG. 4.

### EXAMPLE 5

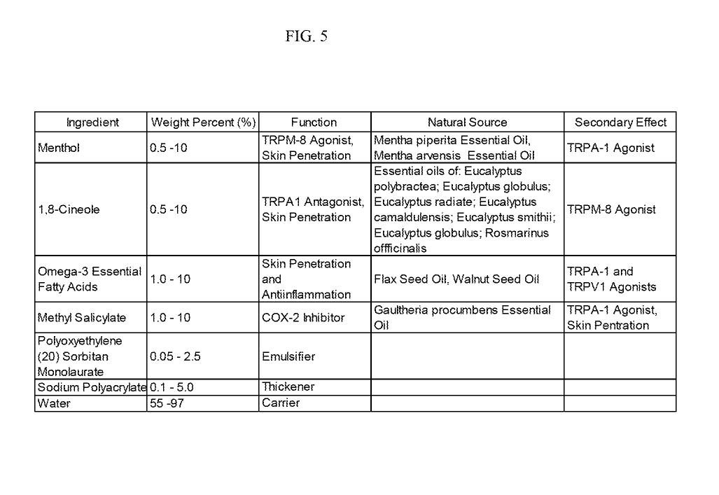

A method of manufacture of the topical analgesic cream containing methyl salicylate, 1,8-cineole, menthol and Omega-3 fatty acids for the composition presented in Example 5 consists of mixing an amount sodium polyacrylate with the oil phase components of the composition to dissolve the sodium polyacrylate, then adding an amount of polyoxyethylene (20) sorbitan monolaurate, followed by then adding an amount of water, such that a stable single phase homogeneous cream results. Mixing is limited to that required to dissolve the sodium polyacrylate with the oil phase components and then to mix the water for a period of time to create the stable single phase homogeneous cream and to minimize volatilization of menthol and 1,8-cineole. Methods of use of the composition of the topical analgesic given in Example 5 include but are not meant to be limited to placing the cream composition in a roll-on bottle then placing the roll-on ball into the roll-on bottle container, placing the cream composition in a squeeze tube container, placing the cream composition in a hand pump bottle container and applying a therapeutic amount of the cream on the skin and wetting a patch with the cream and placing on the skin. The topical analgesic cream composition in Example 5 can optionally be made with borneol or a mixture of 1,8-cineole and borneol in the same total concentration range as 1,8-cineole alone presented in Example 5. The composition of a topical analgesic in the form of a cream with methyl salicylate is shown in FIG. 5.

### EXAMPLE 6

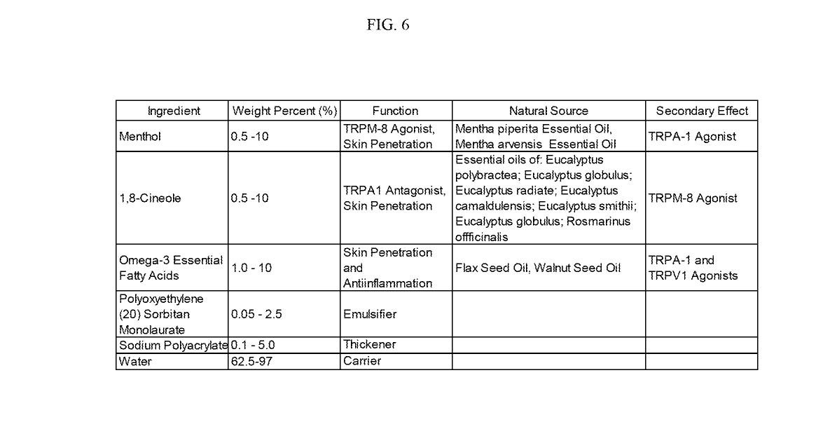

A method of manufacture of the topical analgesic cream containing menthol, 1,8-cineole and Omega-3 fatty acids and not containing methyl salicylate for the composition presented in Example 6 consists of mixing an amount sodium polyacrylate with the oil phase components of the composition to dissolve the sodium polyacrylate, then adding an amount of polyoxyethylene (20) sorbitan monolaurate, followed by then adding an amount of water, such that a stable single phase homogeneous cream results. Mixing is limited to that required to dissolve the sodium polyacrylate with the oil phase components and then to mix the water for a period of time to create the stable single phase homogeneous cream and to minimize volatilization of menthol and 1,8-cineole. Methods of use of the composition of the topical analgesic cream given in Example 6 include but are not meant to be limited to placing she cream composition in a roll-on bottle then placing the roll-on ball into the roll-on bottle container, placing the cream composition in a squeeze tube container, placing the cream composition in a hand pump bottle container and applying a therapeutic amount of the cream on the skin and wetting a patch with the cream and placing on the skin. The topical analgesic cream composition is Example 6 can optionally be made with borneol or a mixture of 1,8-cineole and borneol in the same total concentration range as 1,8-cineole alone presented in Example 6. The composition of a topical analgesic in the form of a cream without methyl salicylate is shown in FIG. 6.

### EXAMPLE 7

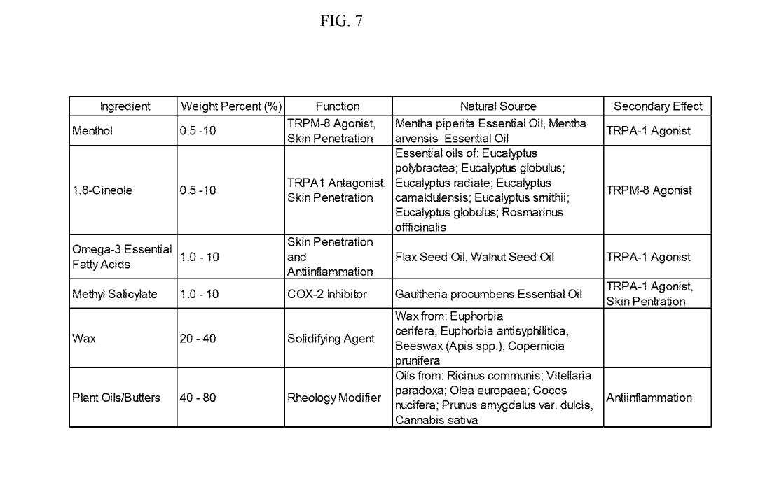

A method of manufacture of the topical analgesic wax containing methyl salicylate, 1,8-cineole, menthol and Omega-3 fatty acids for the composition presented in Example 7 consists of mixing an amount wax with the oil phase components and then heating the mixture to above the boiling point of the wax with the highest boiling point, such that a stable single phase homogeneous hydrophobic solution results. Mixing is limited to that required to melt the wax in the oil phase components for a shortest period of time to create the stable single phase homogeneous cream and to minimize volatilization of menthol and 1,8-cineole and other volatile components of the composition. The rheology of the resultant wax when the melted wax solution is returned to ambient temperatures is controlled by the wax to oily phase material ratio. The melted wax solution can be placed into plastic or metal molds for use in topical analgesic lip balms or wax applicators for use on the skin. The topical analgesic wax composition is Example 7 can optionally be made with borneol or a mixture of 1,8-cineole and borneol in the same total concentration range as 1,8 -cineole alone presented in Example 7. The composition of a topical analgesic in the form of a wax with methyl salicylate is shown in FIG. 7.

### EXAMPLE 8

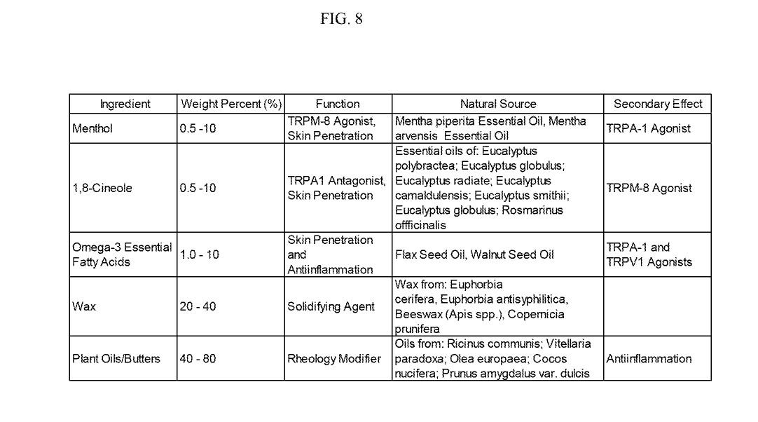

A method of manufacture of the topical analgesic wax containing 1,8-cineole, menthol and Omega-3 fatty acids and not containing methyl salicylate for the composition presented in Example 8 consists of mixing an amount wax with the oil phase components and then heating the mixture to above the boiling point of the wax with the highest boiling point, such that a stable single phase homogeneous hydrophobic solution results. Mixing is limited to that required to melt the wax in the oil phase components for a shortest period of time to create the stable single phase homogeneous cream and to minimize volatilization of menthol and 1,8 -cineole and other volatile components of the composition. The rheology of the resultant wax when the melted wax solution is returned to ambient temperatures is controlled by the wax to oily phase material ratio. The melted wax solution can be placed into plastic or metal molds for use in topical analgesic lip balms or wax applicators for use on the skin. The topical analgesic wax composition in Example 8 can optionally be made with borneol or a mixture of 1,8-cineole and borneol in the same total concentration range as 1,8-cineole alone presented in Example 8. The composition of a topical analgesic in the form of a wax without methyl salicylate is shown in FIG. 8.

### EXAMPLE 9

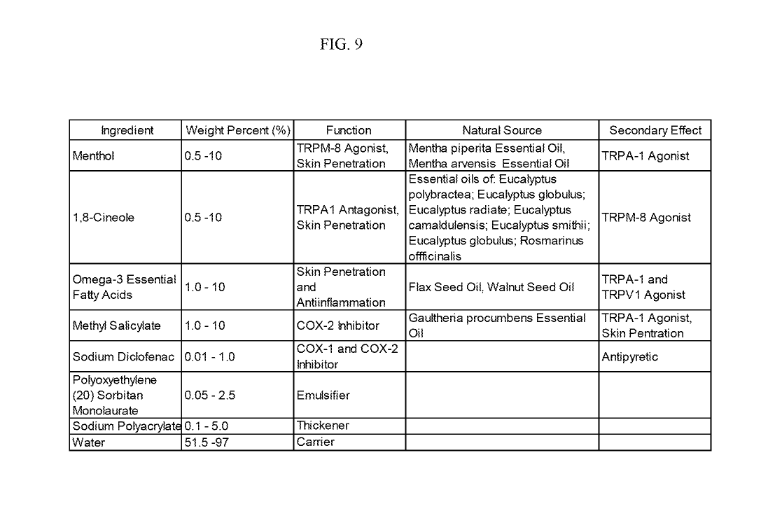

A method of manufacture of the topical analgesic cream containing and sodium diclofenac, methyl salicylate, 1,8-cineole, menthol and Omega-3 fatty acids for the composition presented in Example 9 consists of mixing an amount sodium polyacrylate with the oil phase components of the composition to dissolve the sodium polyacrylate, then adding an amount of polyoxyethylene (20) sorbitan monolaurate, followed by then adding an amount of water that has previously had dissolved into it an amount of sodium diclofenac, such that a stable single phase homogeneous cream results. Mixing is limited to that required to dissolve the sodium polyacrylate with the oil phase components and then to mix the water for a period of time to create the stable single phase homogeneous cream and to minimize volatilization of menthol and 1,8-cineole. Methods of use of the composition of the topical analgesic given in Example 9 include but are not meant to be limited to placing the cream composition in a roll-on bottle then placing the roll-on ball into the roll-on bottle container, placing the cream composition in a squeeze tube container, placing the cream composition in a hand pump bottle container and applying a therapeutic amount of the cream on the skin and wetting a patch with the cream and placing on the skin. Alternatively, the composition presented in Example 9 can also be made without methyl salicylate. The topical analgesic cream composition containing diclofenac composition is Example 9 can optionally be made with borneol or a mixture of 1,8-cineole and borneol is the same total concentration range as 1,8-cineole alone presented in Example 9. The composition of a topical analgesic in the form of a cream with methyl salicylate and Diclofenac is shown in FIG. 9.

### EXAMPLE 10

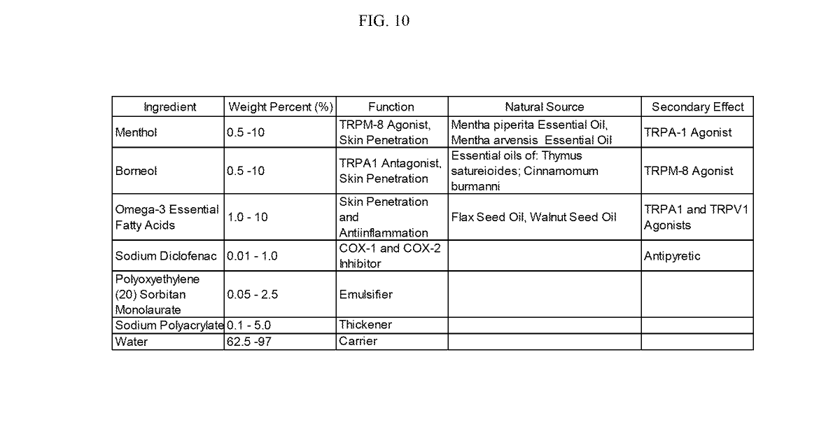

A method of manufacture of the topical analgesic cream containing menthol, borneol, sodium diclofenac and Omega-3 fatty acids for the composition presented in Example 10 consists of mixing an amount sodium polyacrylate with the oil phase components of the composition to dissolve the sodium polyacrylate, then adding an amount of polyoxyethylene (20) sorbitan monolaurate, followed by then adding an amount of water that has previously had dissolved into it an amount of sodium diclofenac, such that a stable single phase homogeneous cream results. Mixing is limited to that required so dissolve the sodium polyacrylate with the oil phase components and then to mix the water for a period of time to create the stable single phase homogeneous cream and to minimize volatilization of menthol and borneol. Methods of use of the composition of the topical analgesic given in Example 9 include but are not meant to be limited to placing the cream composition in a roll-on bottle then placing the roll-on ball into the roll-on bottle container, placing the cream composition in a squeeze tube container, placing the cream composition in a hand pump bottle container and applying a therapeutic amount of the cream on the skin and wetting a patch with the cream and placing on the skin. Alternatively, the composition presented in Example 10 can also be made with 1,-cineole instead of borneol or a mixture of 1,8-cineole and borneol. The composition of a topical analgesic in the form of a cream with borneol is shown in FIG. 10.

### EXAMPLE 11

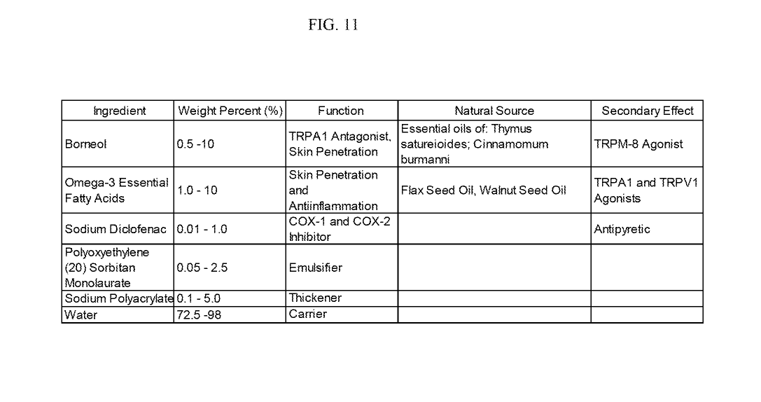

A method of manufacture of the topical analgesic cream containing borneol (as a TRPA1 antagonist and a TRPM8 agonist), sodium diclofenac as a COX-2 inhibitor and Omega-3 fatty acids for the composition presented in Example 11 consists of mixing an amount sodium polyacrylate with the oil phase components, borneol and an oil containing Omega-3 fatty acids, of the composition to dissolve the sodium polyacrylate, then adding an amount of polyoxyethylene (20) sorbitan monolaurate, followed by then adding an amount of water that has previously had dissolved into it an amount of sodium diclofenac such that a stable single phase homogeneous cream results. Mixing is limited to that required to dissolve the sodium polyacrylate with the oil phase components and then to mix the water for a period of time to create the stable single phase homogeneous cream and to minimize volatilization of menthol and borneol. Methods of use of the composition of the topical analgesic given in Example 11 include but are not meant to be limited to placing the cream composition in a roll-on bottle then placing the roll-on ball into the roll-on bottle container, placing the cream composition in a squeeze tube container, placing the cream composition in a hand pump bottle container and applying a therapeutic amount of the cream on the skin and wetting a patch with the cream and placing on the skin. Alternatively, the composition presented in Example 11 can also be made with 1,8-cineole instead of borneol or a mixture of 1,8-cineole and borneol. The composition of a topical analgesic in the form of a cream with borneol is shown in FIG. 11.

### EXAMPLE 12

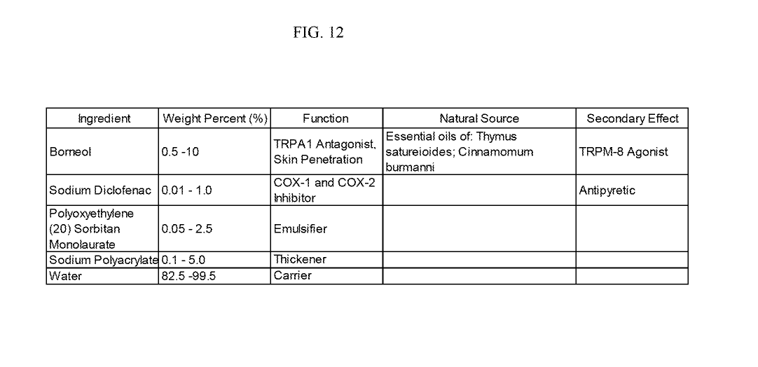

A method of manufacture of the topical analgesic cream containing borneol (as a TRPA1 antagonist and a TRPM8 agonist) and sodium diclofenac as a COX-1 and COX-2 inhibitor for the composition presented in Example 12 consists of mixing an amount sodium polyacrylate with the oil phase components, borneol of the composition to dissolve the sodium polyacrylate, then adding an amount of polyoxyethylene (20) sorbitan monolaurate, followed by then adding an amount of water that has previously had dissolved into it an amount of sodium diclofenac, such that a stable single phase homogeneous cream results. Mixing is limited to that required to dissolve the sodium polyacrylate with the oil phase component and then to mix the water for a period of time to create the stable single phase homogeneous cream and to minimize volatilization of essential oil containing borneol. Methods of use of the composition of the topical analgesic given in Example 12 include but are not meant to be limited to placing the cream composition in a roll-on bottle then placing the roll-on ball into the roll-on bottle container, placing the cream composition in a squeeze tube container, placing the cream composition in a hand pump bottle container and applying a therapeutic amount of the cream on the skin and wetting a patch with the cream and placing on the skin. Alternatively, the composition presented in Example 12 can also be made with 1,8-cineole instead of borneol or a mixture of 1,8-cineole and borneol. The composition of a topical analgesic in the form of a cream with borneol is shown in FIG. 12.

### EXAMPLE 13

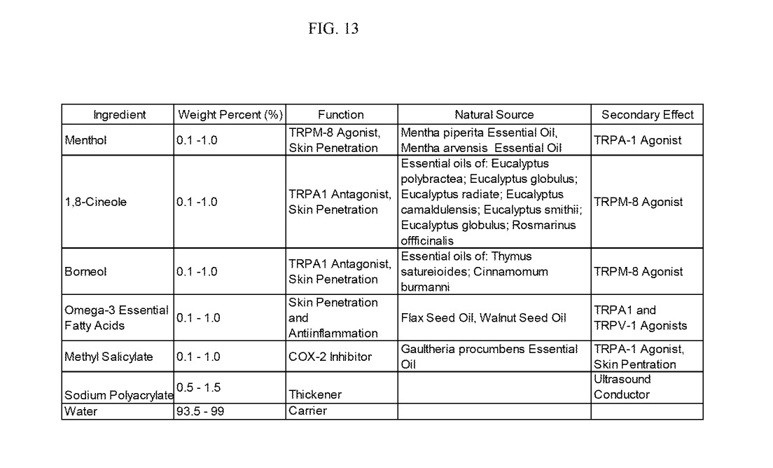

A method of manufacture of the therapeutic ultrasound gel containing borneol and 1,8-cineole (as TRPA1 antagonists and a TRPM8 agonists), menthol as a TRPM8 agonist, methyl salicylate as a COX-2 inhibitor and an oil high in Omega-3 fatty acids for the composition presented in Example 13. This method consists of mixing an amount sodium polyacrylate with the oil phase components to dissolve the sodium polyacrylate, followed by then adding an amount of water such that a stable single phase homogeneous gel results. Mixing is limited to that requited to dissolve the sodium polyacrylate with the oil phase components and then to mix the water for a period of time to create the stable single phase homogeneous gel and to minimize volatilization of essential oil compounds and to minimize the amount air entrained in the gel. Optionally, the water phase can be de-aired by either heating or applying a vacuum prior to use. It is desirable to minimize the entrainment of air in the resulting ultrasound gel for maximum effectiveness as an ultrasound conductive medium. Methods of use of the composition of the topical analgesic given in Example 13 include but are not meant to be limited to placing the ultrasound gel composition in a squeeze tube container, squeezing out a sufficient quantity of the ultrasound gel on an area of skin, then placing the ultrasound transducer on the skin, with the frequency set at the appropriate setting for either diagnostic ultrasound, therapeutic ultrasound, or both. Alternatively, the composition presented in Example 13 can also be made with 1,8-cineole or borneol alone. The composition of a topical analgesic in the form of a gel with borneol is shown in FIG. 13.

### EXAMPLE 14

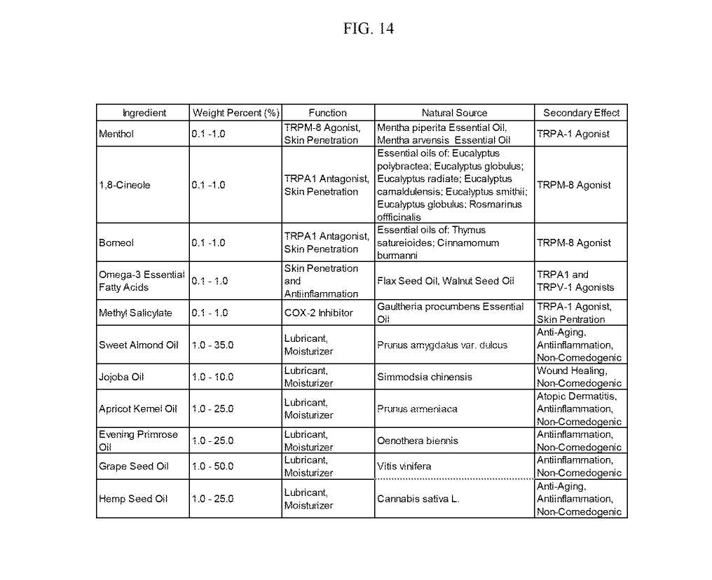

A method oil manufacture of the therapeutic massage oil containing borneol and 1,8-cineole (as TRPA1 antagonists and a TRPM8 agonists), menthol as a TRPM8 agonist, methyl salicylate as a COX-2 inhibitor, an oil high in Omega-3 fatty acids and several fixed oils for the composition presented in Example 14. This method consists of mixing an amount of one or more fixed oils having primary properties of lubricity and moisturizing and secondary properties including non-comedogenic and anti-inflammatory properties and optionally anti-aging, anti-dermatitis properties with the essential oil ingredients providing TRPA1 antagonists, TRPM8 agonists and methyl salicylate as a COX-2. Mixing is limited to that required to create the stable single phase homogeneous oil phase liquid and to minimize volatilization of essential oil compounds and to minimize the amount air entrained in the oil mixture. Methods of use of the composition of the topical analgesic massage oil given in Example 14 include but are not meant to be limited to placing the massage oil composition in a squeeze tube container, squeezing out a sufficient quantity of the massage oil onto the hands of a person providing the massage and onto an area of skin of the person receiving the massage, then massaging the person receiving the massage. Alternatively, the composition presented in Example 14 can also be made without methyl salicylate. The composition of a therapeutic massage oil with borneol is shown in FIG. 14.

### EXAMPLE 15

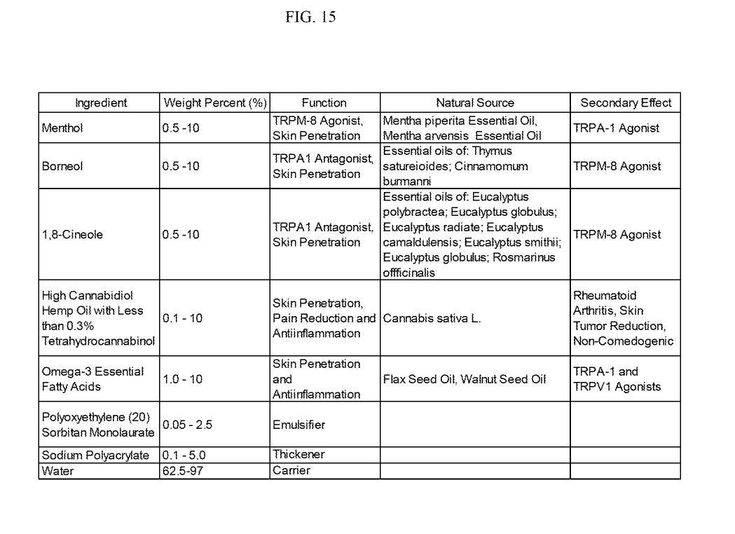

A method of manufacture of the topical analgesic cream containing borneol and 1,8-cineole (as TRPA1 antagonists and a TRPM8 agonists), menthol as a TRPM8 agonist, one or more cannabinoid compounds, preferably a high cannabidiol (CBD)-low tetrahydrocannabinol (THC) Hemp Oil as a pain reliever and anti-inflammatory agent, optionally an oil high in Omega-3 fatty acids, sodium polyacrylate or another suitable thickener for a water-based composition and polyoxyethylene (20) sorbitan monolaurate, or another suitable emulsifying agent, and water as presented in Example 15. This method consists of mixing an amount sodium polyacrylate with the oil phase components comprising, 1,8-cineole, borneol, menthol, optionally Omega-3 fatty acids and Hemp Oil to dissolve the sodium polyacrylate, then adding an amount of polyoxyethylene (20) sorbitan monolaurate, followed by then adding an amount of water and mixing such that a stable single phase homogeneous cream results. Mixing is limited to that required to dissolve the sodium polyacrylate with the oil phase component and then to mix the water for a period of time to create the stable single phase homogeneous cream and to minimize volatilization of essential oil containing 1,8-cineole, borneol and menthol. Methods of use of the composition of the topical analgesic given in Example 15 include but are not meant to be limited to placing the cream composition in a roll-on bottle then placing the roll-on ball into the roll-on bottle container, placing the cream composition in a squeeze tube container, placing the cream composition in a hand pump bottle container and applying a therapeutic amount of the cream on the skin and wetting a patch with the cream and placing on the skin. Alternatively, the composition presented in Example 15 can also be made with 1,-cineole instead of borneol or a mixture of 1,8-cineole and borneol. Optionally, the composition presented in Example 15 can also be manufactured without menthol and optionally without separately added Omega-3 fatty acids. The composition of a topical analgesic in the form of a cream with borneol is shown in FIG. 15.

### EXAMPLE 16

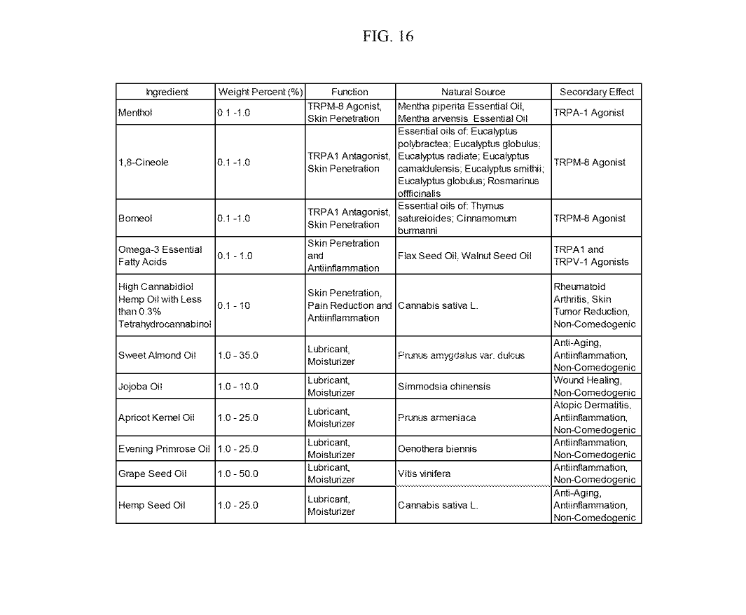

A method of manufacture of the therapeutic massage oil containing borneol and 1,8-cineole (as TRPA1 antagonists and a TRPM8 agonists), menthol as a TRPM8 agonist, one or more cannabinoid compounds, preferably a high cannabidiol (CBD)-low tetrahydrocannabinol (THC) Hemp Oil as a pain reliever and anti-inflammatory agent, optionally an oil high in Omega-3 fatty acids and several fixed oils for the composition presented in Example 16. This method consists of mixing an amount of one or more fixed oils having primary properties of lubricity and moisturizing and secondary properties including non-comedogenic and antiflammatory properties and one or more cannabinoid compounds, preferably a high cannabidiol (CBD)-low tetrahydrocannabinol (THC) Hemp Oil as a pain reliever and anti-inflammatory agent with the essential oil ingredients providing TRPA1 antagonists and TRPM8 agonists. Mixing is limited to that required to create the stable single phase homogeneous oil phase liquid and to minimize volatilization of essential oil compounds and to minimize the amount air entrained in the oil mixture. Methods of use of the composition of the topical analgesic massage oil given in Example 16 include but are not meant to be limited to placing the massage oil composition in a squeeze tube container, squeezing out a sufficient quantity of the massage oil onto the hands of a person providing the massage and onto an area of skin of the person receiving the massage, then massaging the person receiving the massage. Alternatively, the composition presented in Example 16 can also be made with methyl salicylate. Optionally, the composition in Example 16 can be manufactured without menthol. Alternatively, the composition presented in Example 16 can also be made with 1,8-cineole instead of borneol or a mixture of 1,8-cineole and borneol. The composition of a therapeutic massage oil with borneol is shown in FIG. 16.

### EXAMPLE 17

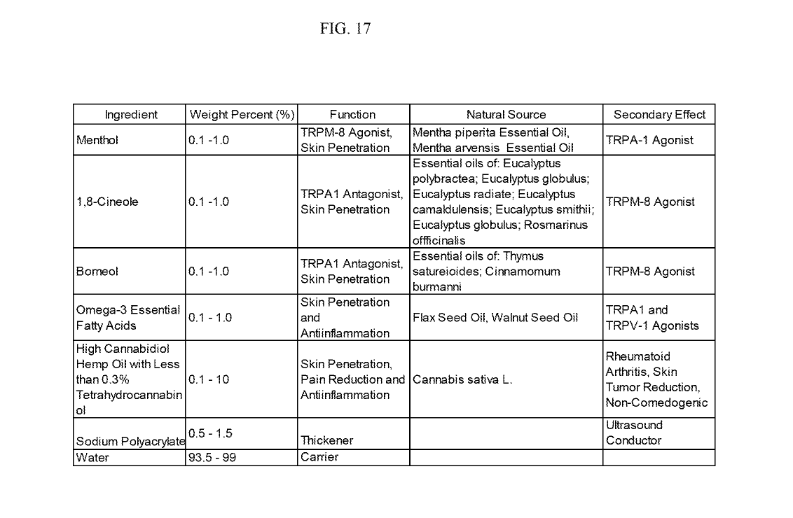

A method of manufacture of the therapeutic ultrasound gel containing borneol and 1,8-cineole (as TRPA1 antagonists and a TRPM8 agonists), menthol as a TRPM8 agonist, one or more cannabinoid compounds, preferably a high cannabidiol (CBD)-low tetrahydrocannabinol (THC) Hemp Oil as a pain reliever and anti-inflammatory agent, and optionally an oil high in Omega-3 fatty acids for the composition presented in Example 17. This method consists of mixing an amount sodium polyacrylate with the oil phase components to dissolve the sodium polyacrylate, followed by then adding an amount of water such that a stable single phase homogeneous gel results. Mixing is limited to that required to dissolve the sodium polyacrylate with the oil phase components and then to mix the water for a period of time to create the stable single phase homogeneous gel and to minimize volatilization of essential oil compounds and to minimize the amount air entrained in the gel. Optionally, the water phase can be de-aired by either heating or applying a vacuum prior to use. It is desirable to minimize the entrainment of air in the resulting ultrasound gel for maximum effectiveness as an ultrasound conductive medium. Methods of use of the composition of the topical analgesic ultrasound gel given in Example 17 include but are not meant to be limited to placing the ultrasound gel composition in a squeeze tube container, squeezing out a sufficient quantity of the ultrasound gel on an area of skin, then placing the ultrasound transducer on the skin, with the frequency set at the appropriate setting for either diagnostic ultrasound, therapeutic ultrasound, or both. Alternatively, the composition presented in Example 17 can also be made with methyl salicylate. Optionally, the composition in Example 17 can be manufactured without menthol. Alternatively, the composition presented in Example 17 can also be made with 1,8-cineole or borneol alone. The composition of a therapeutic ultrasound gel with borneol is shown in FIG. 17.

### EXAMPLE 18

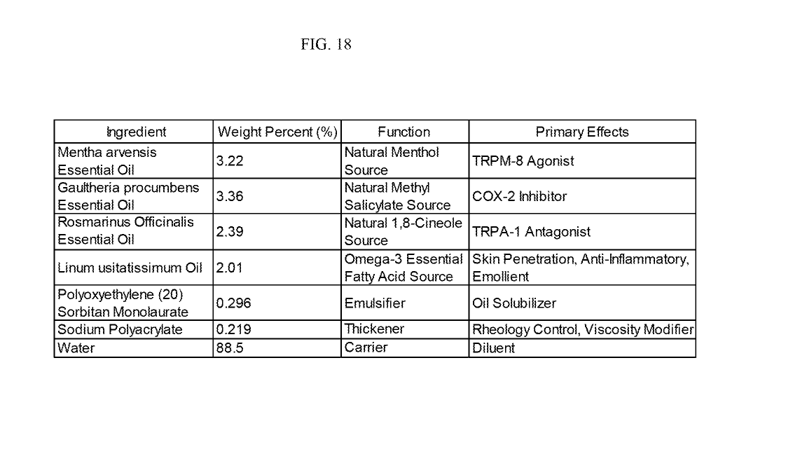

A preferred composition and method of manufacture of the topical analgesic cream containing essential oil sources of methyl salicylate, menthol, 1,8-cineole and flax seed oil is presented in Example 18. Manufacturing consists of mixing 2.19 g sodium polyacrylate with the oil phase components (consisting of 32.2 g Mentha arvensis Essential Oil, 33.6 g. Gaultheria procumbens Essential Oil, 23.9 g Rosmarinus Officinalis Essential Oil, 20.1 g Linum usitatissimum Oil) sufficiently to dissolve the sodium polyacrylate, then adding 2.96 g polyoxyethylene (20) sorbitan monolaurate and then mixing, followed by then adding 885 g water then rapidly mixing for 2 to 5 minutes for the stable single phase homogeneous cream to form. Mixing is limited to that required to dissolve the sodium polyacrylate with the oil phase components and then to mix the water for a period of time to create the stable single phase homogeneous cream and to minimize volatilization of essential oils containing methyl salicylate, menthol and 1,8-cineole. Methods of use of the composition of the topical analgesic cream given in Example 18 include but are not meant to be limited to placing the cream composition in a roll-on bottle then placing the roll-on ball into the roll-on bottle container, placing the cream composition in a squeeze tube container, placing the cream composition in a hand pump bottle container and applying a therapeutic amount of the cream on the skin and wetting a patch with the cream and placing on the skin. The composition of a topical analgesic in the form of a cream with methyl salicylate is shown in FIG. 18.

### EXAMPLE 19

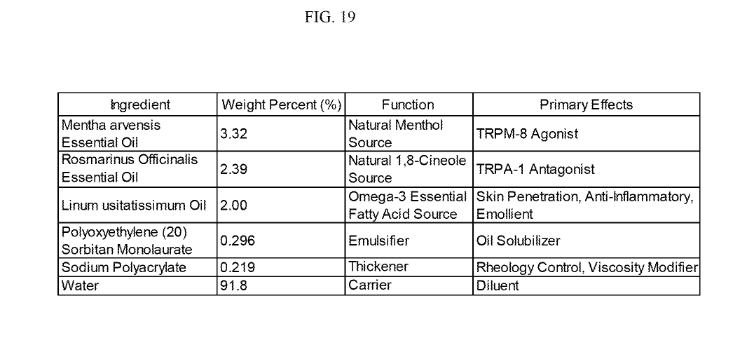

A preferred composition and method of manufacture of the topical analgesic cream containing essential oil sources of menthol and 1,8-cineole and flax seed oil and not containing methyl salicylate is presented in Example 19. Manufacturing consists of mixing 2.19 g sodium polyacrylate with the oil phase components (consisting of 33.2 g Mentha arvensis Essential Oil, 23.9 g Rosmarinus Officinalis Essential Oil, 20.0 g Linum usitatissimum Oil) sufficiently to dissolve the sodium polyacrylate, then adding 2.96 g polyoxyethylene (20) sorbitan monolaurate and then mixing, followed by then adding 918 g water then rapidly mixing for 2 to 5 minutes for the stable single phase homogeneous cream to form. Mixing is limited to that required to dissolve the sodium polyacrylate with the oil phase components and then to mix the water for a period of time to create the stable single phase homogeneous cream and to minimize volatilization of menthol and 1,8-cineole. Methods of use of the composition of the topical analgesic cream given in Example 19 include but are not meant to be limited to placing the cream composition in a roll-on bottle then placing the roll-on ball into the roll-on bottle container, placing the cream composition in a squeeze tube container, placing the cream composition in a hand pump bottle container and applying a therapeutic amount of the cream on the skin and wetting a patch with the cream and placing on the skin. The composition of a topical analgesic in the form of a cream without methyl salicylate is shown in FIG. 19.

### EXAMPLE 20

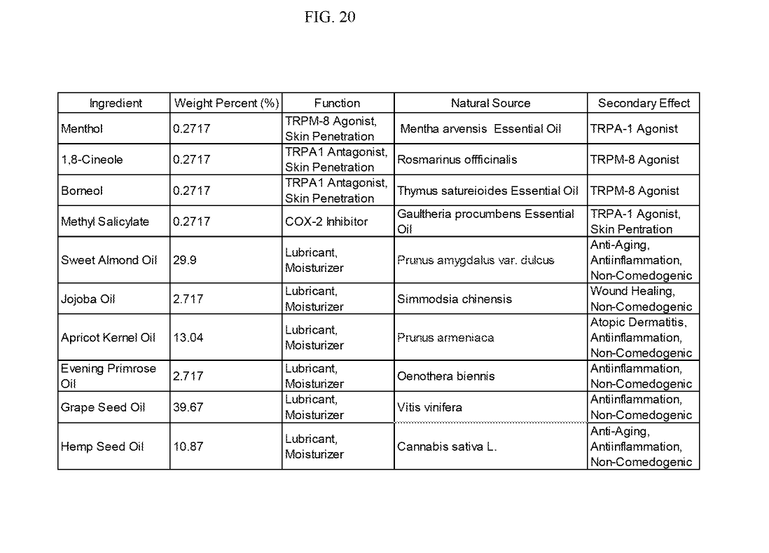

A preferred composition and method of manufacture of a therapeutic massage oil is presented in Example 20. Manufacturing consists of mixing together 2.717 g each of the following; Mentha arvensis Essential Oil, Rosmarinus officinalis, Thymus satureioides Essential Oil and Gaultheria procumbens Essential Oil, then adding 299 g Prunus amygdalus var. dulcus oil, 27.17 g Simmodsia chinensis oil, 130.4 g Prunus armeniaca oil, 27.17 g Oenothera biennis oil, 396.7 g Vitis vinifera seed oil and 108.7 g Cannabis sativa L. seed oil and mixing for 2 minutes to result in a homogeneous oil phase. This method consists of mixing fixed oils first having primary properties of lubricity and moisturizing and secondary properties including non-comedogenic and antiflammatory properties and optionally anti-aging, anti-dermatitis properties with the essential oil ingredients providing TRPA1 antagonists, TRPM8 agonists and method salicylate as a COX-2. Mixing is limited to that required to create the stable single phase homogeneous oil phase liquid and to minimize volatilization of essential oil compounds and to minimize the amount air entrained in the oil mixture. Methods of use of the composition of the topical analgesic massage oil given in Example 20 include but are not meant to be limited to placing the massage oil composition in a squeeze tube container, squeezing out a sufficient quantity of the massage oil onto the hands of a person providing the massage and only an area of skin o the person receiving the massage, then massaging the person receiving the massage. The composition of a therapeutic massage oil is shown in FIG. 20.

### EXAMPLE 21

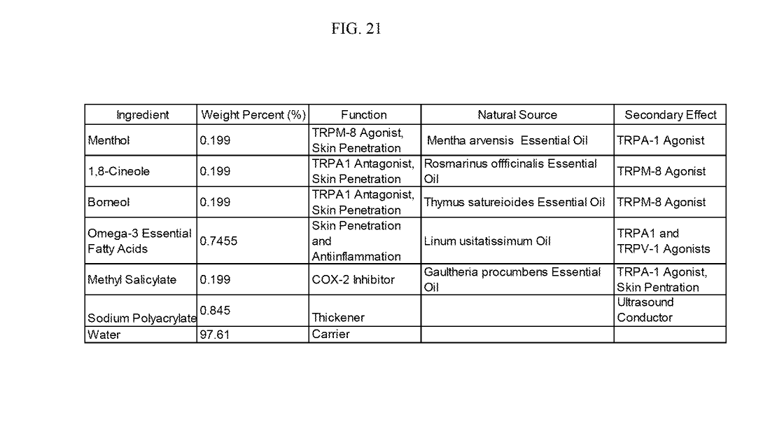

A preferred composition and method of manufacture of a therapeutic ultrasound gel containing is presented in Example 21. Manufacturing consists of mixing together 1.99 g each of the following; Mentha arvensis Essential Oil, Rosmarinus officinalis, Thymus satureioides Essential Oil and Gaultheria procumbens Essential Oil then adding 7.46 g of Linum usitatissimum Oil followed by adding 8.45 g sodium polyacrylate and mixing sufficiently to dissolve the sodium polyacrylate, followed by adding 976.1 g water and mixing for 2 minutes in to result in making a homogeneous gel. Mixing is limited to that required to dissolve the sodium polyacrylate with the oil phase components and then to mix the water for a period of time to create the stable single phase homogeneous gel and to minimize volatilization of essential oil compounds and to minimize the amount air entrained in the gel. Mixing is at a speed and intensity sufficient for dissolution of the oil phase with the oil phase should be just enough to insure dissolution but not beyond this speed and intensity. Methods of use of the composition of the preferred topical analgesic ultrasound gel given in Example 21 include but are not meant to be limited to placing the ultrasound gel composition in a squeeze tube container, squeezing out a sufficient quantity of the ultrasound gel on an area of skin, then placing the ultrasound transducer on the skin, with the frequency set at the appropriate setting for either diagnostic ultrasound, therapeutic ultrasound, or both. The composition of a therapeutic ultrasound gel is shown in FIG. 21.

### EXAMPLE 22

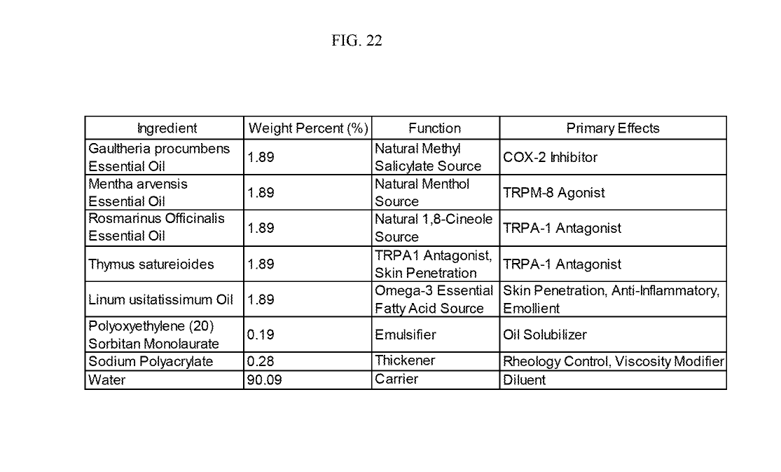

A preferred composition and method of manufacture of the topical analgesic cream containing methyl salicylate, 1,8-cineole, borneol and menthol is presented in Example 22. Manufacturing consists of mixing 2.80 g sodium polyacrylate with the oil phase components (consisting of 18.9 g Mentha arvensis Essential Oil, 18.9 g Gaultheria procumbens Essential Oil, 18.9 g Rosmarinus Officinalis Essential Oil, 18.9 g Thymus satureioides Essential Oil and 18.9 g Linum usitatissimum oil sufficiently to dissolve the sodium polyacrylate, then adding 1.89 g polyoxyethylene (20) sorbitan monolaurate and then mixing, followed by then adding 900.9 g water then rapidly mixing for 2 to 5 minutes for the stable single phase homogeneous cream to form. Mixing is limited to that required to dissolve the sodium polyacrylate with the oil phase components and then to mix the water for a period of time to create the stable single phase homogeneous cream and to minimize volatilization of essential oils containing methyl salicylate, menthol, borneol and 1,8-cineole. Methods of use of the composition of the topical analgesic cream given in Example 22 include but are not meant to be limited to placing the cream composition in a roll-on bottle then placing the roll-on ball into the roll-on bottle container, placing the cream composition in a squeeze tube container, placing the cream composition in a hand pump bottle container and applying a therapeutic amount of the cream on the skin and wetting a patch with the cream and placing on the skin. The composition of a topical analgesic in the form of a cream with methyl salicylate is shown in FIG. 22.

### EXAMPLE 23

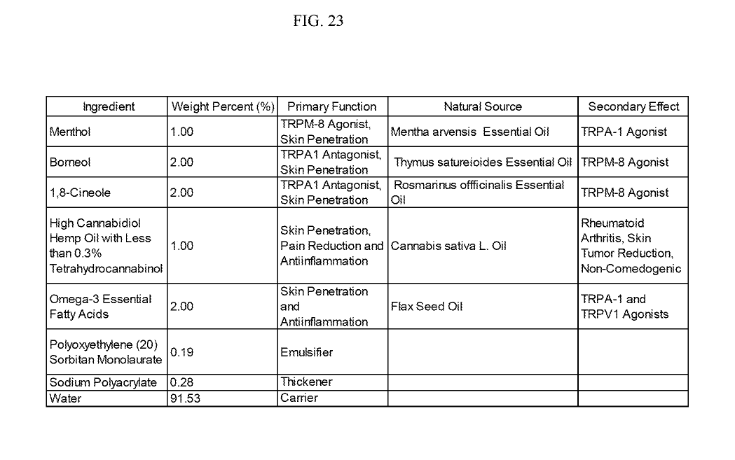

A preferred composition and method of manufacture of the topical analgesic cream containing cannabidiol, 1,8-cineole, borneol and menthol is presented in Example 23. Manufacturing consists of mixing 2.80 g sodium polyacrylate with the oil phase components (consisting of 10.0 g Mentha arvensis Essential Oil, 20.0 g Rosmarinus Officinalis Essential Oil, 20.0 g Thymus satureioides Essential Oil, 10.00 g cannabidiol oil and 20.0 g Linum usitatissimum oil sufficiently to dissolve the sodium polyacrylate, then adding 1.89 g polyoxyethylene (20) sorbitan monolaurate and then mixing, followed by then adding 915.3 g water then rapidly mixing for 2 to 5 minutes for the stable single phase homogeneous cream to form. Mixing is limited to that required to dissolve the sodium polyacrylate with the oil phase components and then to mix the water for a period of time to create the stable single phase homogeneous cream and to minimize volatilization of essential oils containing menthol, borneol and 1,8-cineole. Methods of use of the composition of the topical analgesic cream given in Example 23 include but are not meant to be limited to placing the cream composition in a roll-on bottle then placing the roll-on ball into the roll-on bottle container, placing the cream composition in a squeeze tube container, placing the cream composition in a hand pump bottle container and applying a therapeutic amount of the cream on the skin and wetting a patch with the cream and placing on the skin. The composition of a topical analgesic in the form of a cream with cannabidiol is shown in FIG. 23.

### EXAMPLE 24

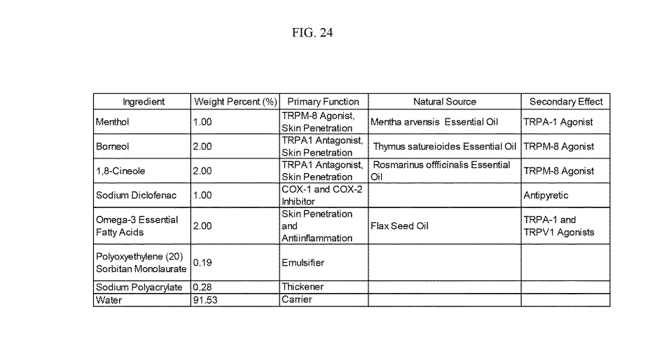

A preferred composition and method of manufacture of the topical analgesic cream containing 1,8-cineole, borneol and menthol is presented in Example 24. Manufacturing consists of mixing 2.80 g sodium polyacrylate with the oil phase components (consisting of 10.0 g Mentha arvensis Essential Oil, 20.0 g Rosmarinus Officinalis Essential Oil, 20.0 g Thymus satureioides Essential Oil and 20.0 g Linum usitatissimum oil sufficiently to dissolve the sodium polyacrylate, then adding 1.89 g polyoxyethylene (20) sorbitan monolaurate and then mixing, followed by then adding 915.3 g water which has previously had 10.00 g sodium diclofenac dissolved into it and then rapidly mixing for 2 to 5 minutes for the stable single phase homogeneous cream to form. Mixing is limited to that required to dissolve the sodium polyacrylate with the oil phase components and then to mix the water for a period of time to create the stable single phase homogeneous cream and to minimize volatilization of essential oils containing menthol, borneol and 1,8-cineole. Methods of use of the composition of the topical analgesic cream given in Example 24 include but are not meant to be limited to placing the cream composition in a roll-on bottle then placing the roll-on ball into the roll-on bottle container, placing the cream composition in a squeeze tube container, placing the cream composition in a hand pump bottle container and applying a therapeutic amount of the cream on the skin and wetting a patch with the cream and placing on the skin. The composition of a topical analgesic in the form of a cream with 1,8-cineole is shown in FIG. 24.

While we have shown and described several embodiments in accordance with our disclosure, it is to be clearly understood that the same may be susceptible to numerous changes apparent to one skilled in the art. Therefore, we do not wish to be limited to the details shown and described but intend to show all changes and modifications that come within the scope of the appended claims.

1. An analgesic composition comprising at least one TRPM8 agonist TRPA1 antagonist, a nonsteroidal anti-inflammatory agent (NSAID), and optionally a carrier.

2. The analgesic composition of claim 1 where the NSAID agent is selected from the group consisting of arthrotec, celecoxib, rofecoxib, vadecoxib, naproxen, ketoprofen, felbinac, ibuprofen, piroxicam, benzydamine, indomethacin and diclofenac.

3. The analgesic composition of claim 1 where the NSAID is diclofenac.

4. The analgesic composition of claim 1 where the TRPM8 agonist is a synthetic compound.

5. The analgesic composition of claim 3 in which the TRPM8 agonist is 1-menthol.

6. The analgesic composition of claim 5 in which 1-menthol is from a synthetic source or is derived from one or more essential oils selected from the group consisting of: Mentha spp., including Mentha piperita; and Mentha arvensis.

7. The analgesic composition of claim 1 in which the TRPM8 agonist is menthone, 1,8-cineole, borneol, linalool, geraniol, or isopulegol.

8. The analgesic composition of claim 1 in which the TRPA1 antagonist is a synthetic compound.

9. The analgesic composition of claim 1 in which the TRPA1 antagonist is 1,8-cineole from a synthetic source or derived from natural sources comprising one or more essential oils selected from the group consisting of: Eucalyptus spp., including Eucalyptus polybractea, Eucalyptus globulus, Eucalyptus radiate, Eucalyptus camaldulensis, Eucalyptus smithii, and Eucalyptus globulus; Rosmarinus spp., including Rosmarinus officinalis; and Salvia lavandulifolia.

10. The analgesic composition of claim 1 in which the TRPA1 antagonist is borneol from a synthetic source or derived from one or more of the essential oils selected from the group consisting of: Thymus satureioides and Cinnamomum burmanni.

11. The analgesic composition of claim 1 in which the the carrier is selected from the group consisting of a paste, a liquid, a gel, a wax, a cream, a suspension, a film, a stick, a patch, a solid, a powder, nanoparticles, and granules; and in which the carrier comprises one or more additives or excipients selected from the group consisting of odorants, deodorants, diluents, fillers, binders, adhesives, disintegrants, lubricants, anti-adhesives glidents, coloring agents, sweeteners, coating agents, plasticizers, wetting agents, and buffers.

12. A method of relieving pains in mammals by administering an analgesic composition comprising of at least one TRPM8 agonist, at least one TRPA1 antagonist, and a nonsteroidal anti-inflammatory agent (NSAID).

13. The method of claim 12 where the NSAID agent is selected from the group consisting of arthrotec, celecoxib, rofecoxib, vadecoxib, naproxen, ketoprofen, felbinac, ibuprofen, piroxicam, benzydamine, indomethacin and diclofenac.

14. The method of claim 12 where the TRPM8 agonist is a synthetic compound.

15. The method of claim 12 in which the TRPM8 agonist is 1-menthol.

16. The method of claim 15 in which the source of 1-menthol is from a synthetic source or selected from one or more essential oils selected from the group consisting of: Mentha spp.; Mentha piperita; and Mentha arvensis.

17. The method of claim 12 in which the TRPM8 agonist is menthone, 1,8-cineole, borneol linalool, geraniol, or isopulegol.

18. The method of claim 12 in which the TRPA1 antagonist is a synthetic compound.

19. The method of claim 12 in which the TRPA1 antagonist is 1,8-cineole from a synthetic source or derived from natural sources comprising one or more essential oils selected from the group consisting of: Eucalyptus spp., including Eucalyptus polybractea; Eucalyptus globulus, Eucalyptus radiate, Eucalyptus camaldulensis, Eucalyptus smithii and Eucalyptus globulus; Rosmarinus spp. including Rosmarinus officinalis; and Salvia lavandulifolia; or is borneol from a synthetic source or derived front one or more of the essential oils selected from the group consisting of: Thymus satureioides and Cinnamomum burmanni.

20. The method of claim 12 wherein the composition is administered orally or topically.
